SEQUENCE LISTING (9 sequences)

SEQ ID NO: 1 - protein, 10 aa, synthetic construct
  REGION 1..10: note = Synthetic: INBRX-109 CDR1
SGLTFPNYGM

SEQ ID NO: 2 - protein, 10 aa, synthetic construct
  REGION 1..10: note = Synthetic: INBRX-109 CDR2
AIYWSGGTVY

SEQ ID NO: 3 - protein, 17 aa, synthetic construct
  REGION 1..17: note = Synthetic: INBRX-109 CDR3
AVTIRGAATQTWKYDYW

SEQ ID NO: 4 - protein, 125 aa, synthetic construct
  REGION 1..125: note = Synthetic: INBRX-109 VHH
EVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGG

SEQ ID NO: 5 - protein, 254 aa, synthetic construct
  REGION 1..254: note = Synthetic: INBRX-109 VHH-linker-VHH
EVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGG

SEQ ID NO: 6 - protein, 224 aa, synthetic construct
  REGION 1..224: note = Synthetic: Fc (with hinge)
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 480 aa, synthetic construct
  REGION 1..480: note = Synthetic: INBRX-109
EVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 440 aa, Homo sapiens
  REGION 1..440: note = misc_feature - Human DR5 (with signal peptide, amino acids 1-55)
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 9 - protein, 385 aa, Homo sapiens
  REGION 1..385: note = misc_feature - Mature human DR5
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

(19) United States
(12) Patent Application Publication
(10) Pub. No.: US 2025/0302948 A1
(43) Pub. Date: Oct. 2, 2025

(54) DR5 Agonist and IAP Antagonist Combination Therapy

(71) Applicant: Inhibrx Biosciences, Inc., La Jolla, CA (US)

(72) Inventors: Katelyn M. Willis, San Diego, CA (US); Monica Holcomb, La Jolla, CA (US); William Crago, San Diego, CA (US); Brendan P. Eckelman, Encinitas, CA (US)

(73) Assignee: Inhibrx Biosciences, Inc., La Jolla, CA (US)

(21) Appl. No.: 18/865,854

(22) PCT Filed: May 22, 2023
(86) PCT No.: PCT/US2023/067279

§ 371 Date: Nov. 14, 2024

Related U.S. Application Data
(60) Provisional application No. 63/344,675, filed on May 23, 2022.
(60) Provisional application No. 63/494,276, filed on Apr. 5, 2023.

(51) Int. Cl. A61K 39/395; A61K 31/404; A61K 31/407; A61K 31/427; A61P 35/00

(52) U.S. Cl. CPC A61K 39/3955; A61K 31/404; A61K 31/407; A61K 31/427; A61P 35/00

(57) ABSTRACT

Provided herein are methods of treating cancer with a combination of a DR5 agonist and an IAP antagonist.

### CROSS-REFERENCE TO RELATED APPLICATIONS

This application claims the benefit of priority of U.S. Provisional Application No. 63/344,675, filed May 23, 2022, and U.S. Provisional Application No. 63/494,276, filed Apr. 5, 2023; each of which is incorporated by reference herein in its entirety for any purpose.

### INCORPORATION BY REFERENCE OF SEQUENCE LISTING

This application incorporates by reference a Sequence Listing submitted with this application in electronic format entitled 01202-0058-00PCT_Sequence_Listing, created May 5, 2023, which is 14,058 bytes in size.

### FIELD

The present invention relates to treatment of cancer with a combination of a DR5 agonist and an IAP antagonist.

### BACKGROUND

Death Receptor 5 (DR5; also known as TNFRSF10B or TRAILR2) is a member of the TNF receptor superfamily (TNFRSF) and a cell surface receptor of the TNF-receptor superfamily that binds TNF-related apoptosis-inducing ligand (TRAIL). TRAIL evolved to play critical roles in mammalian development and host defense by selectively eradicating unwanted, infected and malignant cells from healthy cell populations. On binding the TNF receptor family members DR4 or DR5, TRAIL induces cell death via caspase-dependent apoptosis. DR5 appears to be the primary receptor on tumor cells that facilitates the observed tumor biased activity of the TRAIL pathway. DR5 is activated by the natural ligand TRAIL, which brings three DR5 receptors within close proximity thereby activating intracellular caspase-8 and initiating activation of other death-inducing caspases, such as caspases-9 and caspases-3. Thus, initiation of this cell death pathway requires clustering of DR5 receptors for efficient cell death. DR5 agonists are promising therapeutic candidates for treating cancer.

Inhibitor of Apoptosis Proteins (IAPs) are a class of proteins that negatively regulate caspases and apoptosis. IAPs are frequently dysregulated in many cancers and have been suggested as contributing to apoptosis resistance in cancer cells. IAPs contribute to tumor cell survival, chemotherapy resistance, disease progression, and poor prognosis. In addition, IAPs play an important role in immune regulation. Examples of IAPs include cellular-IAP1 (cIAP1), cellular-IAP2 (cIAP2), and X-linked inhibitor of apoptosis protein (XIAP). Due to their significant biological functions in cell death and immune responses, IAPs have been emerging as drug targets in a wide range of malignancies. Several IAP antagonists have been developed and their clinical benefits are currently being investigated. For example, the second mitochondria-derived activator of caspases (SMAC) protein inhibits IAP function, and multiple small molecules mimicking SMAC function (known as SMAC mimetics) have been developed.

### SUMMARY

Provided herein are methods of treating cancer in a subject with a Death Receptor 5 (DR5) agonist and an Inhibitor of Apoptosis Protein (IAP) antagonist. In some embodiments, the methods comprise administering a multivalent Death Receptor 5 (DR5)-binding polypeptide and an Inhibitor of Apoptosis Protein (IAP) antagonist. In some embodiments, the multivalent DR5-binding polypeptide is at least tetravalent. In some embodiments, the multivalent DR5-binding polypeptide is tetravalent.
Embodiment 1. A method of treating cancer in a subject in need thereof, comprising administering to the subject (a) a Death Receptor 5 (DR5) agonist, wherein the DR5 agonist is tetravalent, and (b) an Inhibitor of Apoptosis Protein (IAP) antagonist.
Embodiment 2. The method of embodiment 1, wherein the DR5 agonist is a DR5-binding polypeptide.
Embodiment 3. The method of embodiment 1 or embodiment 2, wherein the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.
Embodiment 4. The method of embodiment 3, wherein the at least one VHH domain comprises an amino acid sequence at least 90%, at least 95%, at least 99%, or 100% identical to the amino acid sequence of SEQ ID NO: 4.
Embodiment 5. The method of any one of embodiments 2-4, wherein the DR5-binding polypeptide comprises a VHH domain comprising the amino acid sequence of SEQ ID NO: 4.
Embodiment 6. The method of any one of embodiments 2-5, wherein the DR5-binding polypeptide comprises an Fc region.
Embodiment 7. The method of embodiment 6, wherein the Fc region comprises the amino acid sequence of SEQ ID NO: 6.
Embodiment 8. The method of any one of embodiments 2-7, wherein the DR5-binding polypeptide has the structure VHH-linker-VHH-linker-Fc.
Embodiment 9. The method of any one of embodiments 2-8, wherein each VHH domain comprises a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.
Embodiment 10. The method of any one of embodiments 2-9, wherein the VHH-linker-VHH comprises an amino acid sequence at least 90%, at least 95%, at least 99%, or 100% identical to the amino acid sequence of SEQ ID NO: 5.
Embodiment 11. The method of embodiment 10, wherein the VHH-linker-VHH comprises the amino acid sequence of SEQ ID NO: 5.
Embodiment 12. The method of any one of embodiments 2-11, wherein the DR5-binding polypeptide comprises an amino acid sequence at least 90%, at least 95%, at least 99%, or 100% identical to the amino acid sequence of SEQ ID NO: 7.
Embodiment 13. The method of any one of embodiments 2-12, wherein the DR5-binding polypeptide comprises the amino acid sequence of SEQ ID NO: 7.
Embodiment 14. The method of any one of embodiments 2-12, wherein the DR5-binding polypeptide consists of the amino acid sequence of SEQ ID NO: 7.
Embodiment 15. The method of embodiment 1 or embodiment 2, wherein the DR5 agonist is INBRX-109.
Embodiment 16. The method of any one of embodiments 1-15, wherein the IAP antagonist is a small molecule.
Embodiment 17. The method of any one of embodiments 1-16, wherein the IAP antagonist is APG-1387 (Ascentage Pharma Group International), birinapant (IGM Biosciences, Inc.), AZD5582 (AstraZeneca), LCL161 (Novartis), Debio 1143 (Merck, Debiopharm), or ASTX660 (Astex Pharmaceuticals, Inc.).
Embodiment 18. The method of embodiment 17, wherein the IAP antagonist is APG-1387.
Embodiment 19. The method of embodiment 17, wherein the IAP antagonist is birinapant.
Embodiment 20. The method of embodiment 17, wherein the IAP antagonist is Debio 1143.
Embodiment 21. The method of embodiment 17, wherein the IAP antagonist is LCL161.
Embodiment 22. The method of any one of embodiments 1-21, wherein the DR5 agonist and the IAP antagonist are administered separately.
Embodiment 23. The method of embodiment 22, wherein the DR5 agonist and the IAP antagonist are administered sequentially.
Embodiment 24. The method of embodiment 22 or 23, wherein at least one dose, or the first dose, of the DR5 agonist is administered before the IAP antagonist.
Embodiment 25. The method of embodiment 22 or 23, wherein at least one dose, or the first dose, of the DR5 agonist is administered after the IAP antagonist.
Embodiment 26. The method of any one of embodiments 1-21, wherein the DR5 agonist and the IAP antagonist are administered concurrently.
Embodiment 27. The method of any one of embodiments 1-26, wherein the DR5 agonist and the IAP antagonist act synergistically.
Embodiment 28. The method of embodiment 27, wherein synergy is determined in an in vitro cell survival assay.
Embodiment 29. The method of any one of embodiments 1-28, wherein administration of the DR5 agonist and the IAP antagonist results in a synergistic effect, compared to each agent administered alone.
Embodiment 30. The method of any one of embodiments 1-29, wherein the cancer is adrenal cancer; astrocytoma; basal cell carcinoma, biliary tract cancer; bladder cancer; bone cancer; brain and central nervous system cancer; breast cancer; cancer of the peritoneum; cervical cancer; choriocarcinoma; chondrosarcoma; Ewing sarcoma; colon and rectum cancer (colorectal cancer); connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; leukemia; liver cancer; lung cancer; small-cell lung cancer; non-small cell lung cancer; adenocarcinoma of the lung; squamous carcinoma of the lung; melanoma; myeloma; neuroblastoma; oral cavity cancer (lip, tongue, mouth, and/or pharynx); ovarian cancer; pancreatic cancer, such as pancreatic adenocarcinoma; pituitary gland cancer; prostate cancer; retinoblastoma; rhabdomyosarcoma; rectal cancer; cancer of the respiratory system; mesothelioma; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.
Embodiment 31. A DR5 agonist for use in a method of treating cancer, wherein the method comprises administering the DR5 agonist in combination with an IAP antagonist.
Embodiment 32. Use of a DR5 agonist for the manufacture of a medicament for treating cancer, wherein the medicament is for administration with an IAP antagonist.

### BRIEF DESCRIPTION OF THE DRAWINGS

FIG. 1A-1E shows results of experiments in which six different cancer cell lines were contacted with INBRX-109 (1 nM), an IAP antagonist, or a combination of INBRX-109 (1 nM) and an IAP antagonist. The bar graphs show the number of cell lines (out of six tested) that are resistant (0-25% cytotoxicity), intermediate (25-75% cytotoxicity), or sensitive (75-100% cytotoxicity). The six cell lines tested were: LOVO, LS174T, SW620, SNU-C2B, HT-29, and LS411N. The number of cancer cell lines is shown on the y-axis of each bar graph. The results are shown for IAP antagonists ASTX660 (900 nM) (FIG. 1A), AZD5582 (450 nM) (FIG. 1B), birinapant (5 μM) (FIG. 1C), Debio 1143 (4.5 μM) (FIG. 1D), and LCL-161 (5 μM) (FIG. 1E).

FIG. 2A-2B shows results of an LCL-161 titration experiment in which cancer cell lines were contacted with different concentrations of LCL-161, alone or in combination with 1 nM INBRX-109. Percent survival of cancer cells is shown on the y-axis of each graph. The results are shown for cancer cell lines LS174T (FIG. 2A) and SW620 (FIG. 2B). The dotted line labeled “1 nM INBRX-109 only” shows the percent survival of cancer cells treated with 1 nM INBRX-109 alone.

FIG. 3A-3B shows results of a birinapant titration experiment in which cancer cell lines were contacted with different concentrations of birinapant, alone or in combination with 1 nM INBRX-109. Percent survival of cancer cells is shown on the y-axis of each graph. The results are shown for cancer cell lines LS174T (FIG. 3A) and SW620 (FIG. 3B). The dotted line labeled “1 nM INBRX-109 only” shows the percent survival of cancer cells treated with 1 nM INBRX-109 alone.

FIG. 4A-4L shows results of a Debio-1143 titration experiment in which cancer cell lines were contacted with different concentrations of Debio-1143, alone or in combination with 1 nM INBRX-109. The results are shown for cancer cell lines A253 (FIG. 4A), CAL-27 (FIG. 4B), COLO-680N (FIG. 4C), Detroit 562 (FIG. 4D), FADU (FIG. 4E), KYSE-70 (FIG. 4F), SCC-9 (FIG. 4G), SCC-25 (FIG. 4H), T.Tn (FIG. 4I), TE-1 (FIG. 4J), KYSE-270 (FIG. 4K). Percent survival of cancer cells is shown on the y-axis of each graph. The dotted line labeled “1 nM INBRX-109 only” shows the percent survival of cancer cells treated with 1 nM INBRX-109 alone. The box and whiskers plot (FIG. 4L) shows the % cytotoxicity (% cell death) for all 18 cell lines of Table 8 treated with INBRX-109 and Debio-1143 in combination.

FIG. 5 shows results of a birinapant titration experiment in which breast cancer cell lines were contacted with different concentrations of birinapant, alone or in combination with 1 nM INBRX-109. The box and whiskers plot shows the % cytotoxicity (% cell death) for 13 cell lines of Table 9 treated with INBRX-109 or birinapant alone, or INBRX-109 and birinapant in combination.

FIG. 6A-6G show results of an IAP antagonist titration experiment in which cancer cell lines were contacted with different concentrations of birinapant or Debio 1143, alone or in combination with 1 nM INBRX-109. The box and whiskers plots in FIG. 6A-6D show the % cytotoxicity (% cell death) for chondrosarcoma (FIG. 6A), pancreatic (FIG. 6B), lung cancer (FIG. 6C), and the 16 cell lines listed in Table 10A (FIG. 6D) treated with INBRX-109 or birinapant alone, or INBRX-109 and birinapant in combination in these studies. The box and whiskers plots in FIG. 6E-6G show the % cytotoxicity (% cell death) for pancreatic (FIG. 6E), lung cancer (FIG. 6F), and the 12 cell lines listed in Table 10B (FIG. 6G) treated with INBRX-109 or Debio 1143 alone, or INBRX-109 and Debio 1143 in combination in these studies.

### DETAILED DESCRIPTION

Embodiments provided herein relate to methods of treating cancer with a combination of a Death Receptor 5 (DR5) agonist and an IAP (Inhibitor of Apoptosis Protein) antagonist.

### Definitions and Various Embodiments

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an immunoglobulin heavy chain is that of the EU index as in Kabat et al., Sequences of Proteins of Immunological Interest, 5^{th} Ed. Public Health Service, National Institutes of Health, Bethesda, Md. (1991). The “EU index as in Kabat” refers to the residue numbering of the human IgG1 EU antibody.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The phrase “reference sample”, “reference cell”, or “reference tissue”, denote a sample with at least one known characteristic that can be used as a comparison to a sample with at least one unknown characteristic. In some embodiments, a reference sample can be used as a positive or negative indicator. A reference sample can be used to establish a level of protein and/or mRNA that is present in, for example, healthy tissue, in contrast to a level of protein and/or mRNA present in the sample with unknown characteristics. In some embodiments, the reference sample comes from the same subject, but is from a different part of the subject than that being tested. In some embodiments, the reference sample is from a tissue area surrounding or adjacent to the cancer. In some embodiments, the reference sample is not from the subject being tested, but is a sample from a subject known to have, or not to have, a disorder in question (for example, a particular cancer or DR5-related disorder). In some embodiments, the reference sample is from the same subject, but from a point in time before the subject developed cancer. In some embodiments, the reference sample is from a benign cancer sample, from the same or a different subject. When a negative reference sample is used for comparison, the level of expression or amount of the molecule in question in the negative reference sample will indicate a level at which one of skill in the art will appreciate, given the present disclosure, that there is no and/or a low level of the molecule. When a positive reference sample is used for comparison, the level of expression or amount of the molecule in question in the positive reference sample will indicate a level at which one of skill in the art will appreciate, given the present disclosure, that there is a level of the molecule.

The terms “benefit”, “clinical benefit”, “responsiveness”, and “therapeutic responsiveness” as used herein in the context of benefiting from or responding to administration of a therapeutic agent, can be measured by assessing various endpoints, e.g., inhibition, to some extent, of disease progression, including slowing down and complete arrest; reduction in the number of disease episodes and/or symptoms; reduction in lesion size; inhibition (that is, reduction, slowing down or complete stopping) of disease cell infiltration into adjacent peripheral organs and/or tissues; inhibition (that is, reduction, slowing down or complete stopping) of disease spread; relief, to some extent, of one or more symptoms associated with the disorder; increase in the length of disease-free presentation following treatment, for example, progression-free survival; increased overall survival; higher response rate; and/or decreased mortality at a given point of time following treatment. A subject or cancer that is “non-responsive” or “fails to respond” is one that has failed to meet the above noted qualifications to be “responsive”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The terms “DR5,” “death receptor 5,” “TNFRSF10B,” and “TRAILR2” as used herein refer to any native, mature DR5 that results from processing of a DR5 precursor in a cell. The term includes DR5 from any vertebrate source, including mammals such as primates (e.g., humans and cynomolgus or rhesus monkeys) and rodents (e.g., mice and rats), unless otherwise indicated. The term also includes naturally-occurring variants of DR5, such as splice variants or allelic variants. A nonlimiting exemplary precursor human DR5 amino acid sequence is shown, e.g., in NCBI Accession No. NP_003833.4. See SEQ ID NO: 8. A nonlimiting exemplary precursor human DR5 amino acid sequence is shown, e.g., in SEQ ID NO: 9.

The terms “IAP” and “Inhibitor of Apoptosis Protein” refer to any native, mature proteins in the family of IAP proteins, of which there are at least eight known members. The terms include IAPs from any vertebrate source, including mammals such as primates (e.g., humans and cynomolgus or rhesus monkeys) and rodents (e.g., mice and rats), unless otherwise indicated. The term also includes naturally-occurring variants of IAPs, such as splice variants or allelic variants. IAPs function as key regulators of caspase activity and are defined by the presence of at least one Baculoviral IAP Repeat (BIR) domain. These approximately 70-residue zinc-binding domains enable their interaction with, and suppression of, caspases, and therefore facilitate the inhibition of apoptosis. Most IAPs also contain a RING finger E3 ligase domain at the C-terminus, enabling these proteins to participate in diverse cellular processes, including signal transduction events that promote inflammation, cell cycle progression and migration.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to a DR5 epitope is a sdAb or VHH-containing polypeptide that binds this epitope with greater affinity, avidity, more readily, and/or with greater duration than it binds to other DR5 epitopes or non-DR5 epitopes. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

As used herein, the term “epitope” refers to a site on a target molecule (for example, an antigen, such as a protein, nucleic acid, carbohydrate or lipid) to which an antigen-binding molecule (for example, a sdAb or VHH-containing polypeptide) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between a residue of the antigen-binding molecule and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antigen-binding molecule. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

The term “antibody” is used in the broadest sense and encompass various polypeptides that comprise antibody-like antigen-binding domains, including but not limited to conventional antibodies (typically comprising at least one heavy chain and at least one light chain), single-domain antibodies (sdAbs, comprising at least one VHH domain and an Fc region), VHH-containing polypeptides (polypeptides comprising at least one VHH domain), and fragments of any of the foregoing so long as they exhibit the desired antigen-binding activity. In some embodiments, an antibody comprises a dimerization domain. Such dimerization domains include, but are not limited to, heavy chain constant domains (comprising CH1, hinge, CH2, and CH3, where CH1 typically pairs with a light chain constant domain, CL, while the hinge mediates dimerization) and Fc regions (comprising hinge, CH2, and CH3, where the hinge mediates dimerization).

The term antibody also includes, but is not limited to, chimeric antibodies, humanized antibodies, and antibodies of various species such as camelid (including llama), shark, mouse, human, cynomolgus monkey, etc.

The term “antigen-binding domain” as used herein refers to a portion of an antibody sufficient to bind antigen. In some embodiments, an antigen binding domain of a conventional antibody comprises three heavy chain CDRs and three light chain CDRs. Thus, in some embodiments, an antigen binding domain comprises a heavy chain variable region comprising CDR1-FR2-CDR2-FR3-CDR3, and any portions of FR1 and/or FR4 required to maintain binding to antigen, and a light chain variable region comprising CDR1-FR2-CDR2-FR3-CDR3, and any portions of FR1 and/or FR4 required to maintain binding to antigen. In some embodiments, an antigen-binding domain of an sdAb or VHH-containing polypeptide comprises three CDRs of a VHH domain. Thus, in some embodiments, an antigen binding domain of an sdAb or VHH-containing polypeptide comprises a VHH domain comprising CDR1-FR2-CDR2-FR3-CDR3, and any portions of FR1 and/or FR4 required to maintain binding to antigen.

The term “VHH” or “VHH domain” or “VHH antigen-binding domain” as used herein refers to the antigen-binding portion of a single-domain antibody, such as a camelid antibody or shark antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains antigen binding and specificity.

The terms “single domain antibody” and “sdAb” are used interchangeably herein to refer to an antibody comprising at least one monomeric domain, such as a VHH domain, without a light chain, and an Fc region. In some embodiments, an sdAb is a dimer of two polypeptides wherein each polypeptide comprises at least one VHH domain and an Fc region. As used herein, the terms “single domain antibody” and “sdAb” encompass polypeptides that comprise multiple VHH domains, such as a polypeptide having the structure VHH₁-VHH₂-Fc or VHH₁-VHH₂-VHH₃-Fc, wherein VHH₁, VHH₂, and VHH₃ may be the same or different.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc region. In some such embodiments, the VHH-containing polypeptide may be referred to as an sdAb. Further, in some such embodiments, the VHH polypeptide may form a dimer. Nonlimiting structures of VHH-containing polypeptides, which are also sdAbs, include VHH₁-Fc, VHH₁-VHH₂-Fc, and VHH₁-VHH₂-VHH₃-Fc, wherein VHH₁, VHH₂, and VHH₃ may be the same or different. In some embodiments of such structures, one VHH may be connected to another VHH by a linker, or one VHH may be connected to the Fc by a linker. In some such embodiments, the linker comprises 1-20 amino acids, preferably 1-20 amino acids predominantly composed of glycine and, optionally, serine. In some embodiments, when a VHH-containing polypeptide comprises an Fc, it forms a dimer. Thus, the structure VHH₁-VHH₂-Fc, if it forms a dimer, is considered to be tetravalent (i.e., the dimer has four VHH domains). Similarly, the structure VHH₁-VHH₂-VHH₃-Fc, if it forms a dimer, is considered to be hexavalent (i.e., the dimer has six VHH domains).

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3.

The term “heavy chain constant region” as used herein refers to a region comprising at least three heavy chain constant domains, CH1, hinge, CH2, and CH3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an ai constant region) and IgA2 (comprising an α₂ constant region) antibodies; and IgM antibodies include, but are not limited to, IgM1 and IgM2.

A “Fc region” as used herein refers to a portion of a heavy chain constant region comprising CH2 and CH3. In some embodiments, an Fc region comprises a hinge, CH2, and CH3. In various embodiments, when an Fc region comprises a hinge, the hinge mediates dimerization between two Fc-containing polypeptides. An Fc region may be of any antibody heavy chain constant region isotype discussed herein. In some embodiments, an Fc region is an IgG1, IgG2, IgG3, or IgG4.

An “acceptor human framework” as used herein is a framework comprising the amino acid sequence of a heavy chain variable domain (V_{H}) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all of the human frameworks in a single antigen binding domain, such as a VHH.

“Affinity” refers to the strength of the sum total of noncovalent interactions between a single binding site of a molecule (for example, an antibody, such as an sdAb, or VHH-containing polypeptide) and its binding partner (for example, an antigen). The affinity or the apparent affinity of a molecule X for its partner Y can generally be represented by the dissociation constant (K_{D}) or the K_{D-apparent}, respectively. Affinity can be measured by common methods known in the art (such as, for example, ELISA K_{D}, KinExA, flow cytometry, and/or surface plasmon resonance devices), including those described herein. Such methods include, but are not limited to, methods involving BIAcore®, Octet®, or flow cytometry.

The term “K_{D}”, as used herein, refers to the equilibrium dissociation constant of an antigen-binding molecule/antigen interaction. When the term “K_{D}” is used herein, it includes K_{D} and K_{D-apparent}.

In some embodiments, the K_{D} of the antigen-binding molecule is measured by flow cytometry using an antigen-expressing cell line and fitting the mean fluorescence measured at each antibody concentration to a non-linear one-site binding equation (Prism Software graphpad). In some such embodiments, the K_{D} is K_{D-apparent}.

The term “biological activity” refers to any one or more biological properties of a molecule (whether present naturally as found in vivo, or provided or enabled by recombinant means). Biological properties include, but are not limited to, binding a ligand, inducing or increasing cell proliferation, and inducing or increasing expression of cytokines.

An “agonist” or “activating” antibody or polypeptide is one that increases and/or activates a biological activity of its target antigen. In some embodiments, the agonist antibody or polypeptide binds to an antigen and increases its biologically activity by at least about 20%, 40%, 60%, 80%, 85% or more.

An “antagonist”, a “blocking” or “neutralizing” antibody is one that inhibits, decreases and/or inactivates a biological activity of the target antigen. In some embodiments, the neutralizing antibody binds to an antigen and reduces its biologically activity by at least about 20%, 40%, 60%, 80%, 85% 90%, 95%, 99% or more.

An “affinity matured” sdAb or VHH-containing polypeptide refers to a sdAb or VHH-containing polypeptide with one or more alterations in one or more CDRs compared to a parent sdAb or VHH-containing polypeptide that does not possess such alterations, such alterations resulting in an improvement in the affinity of the sdAb or VHH-containing polypeptide for antigen.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to further refine and optimize sdAb VHH-containing polypeptide performance. In some embodiments, a humanized sdAb or VHH-containing polypeptide comprises a human Fc region. As will be appreciated, a humanized sequence can be identified by its primary sequence and does not necessarily denote the process by which the antibody was created.

An “effector-positive Fc region” possesses an “effector function” of a native sequence Fc region. Exemplary “effector functions” include Fc receptor binding; Clq binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. For example, the term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18 (12): 592-598 (1997); Ghetie et al., Nature Biotechnology, 15 (7): 637-640 (1997); Hinton et al., J. Biol. Chem. 279 (8): 6213-6216 (2004); WO 2004/92219 (Hinton et al.).

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

A “disease” or “disorder” as used herein refers to a condition where treatment is needed and/or desired.

The term “tumor cell”, “cancer cell”, “cancer”, “tumor”, and/or “neoplasm”, unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases.

The terms “cancer” and “tumor” encompass solid and hematological/lymphatic cancers and also encompass malignant, pre-malignant, and benign growth, such as dysplasia. Exemplary cancers include, but are not limited to: adrenal cancer; astrocytoma; basal cell carcinoma; biliary tract cancer; bladder cancer; bone cancer; brain and central nervous system cancer; breast cancer; cancer of the peritoneum; cervical cancer; choriocarcinoma; chondrosarcoma, Ewing sarcoma, colon and rectum cancer (colorectal cancer); connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer, such as pancreatic adenocarcinoma; pituitary gland cancer; prostate cancer; retinoblastoma; rhabdomyosarcoma; rectal cancer; cancer of the respiratory system; mesothelioma; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments, an “increase” or “decrease” refers to a statistically significant increase or decrease, respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of a test agent. This can be determined in any suitable manner and/or using any suitable assay known per se or described herein, depending on the target involved.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering a therapeutic agent. “Ameliorating” also includes shortening or reduction in duration of a symptom.

The term “anti-cancer agent” is used herein in its broadest sense to refer to agents that are used in the treatment of one or more cancers. Exemplary classes of such agents in include, but are not limited to, chemotherapeutic agents, anti-cancer biologics (such as cytokines, receptor extracellular domain-Fc fusions, and antibodies), radiation therapy, CAR-T therapy, therapeutic oligonucleotides (such as antisense oligonucleotides and siRNAs) and oncolytic viruses.

The terms “synergistic,” “synergistically,” and “synergy,” as used herein refer to a more than additive effect of two or more agents. A determination of a synergistic effect between a DR5 agonist and an IAP antagonist may be carried out using the assays described herein.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” or “reference” refers to a composition known to not contain an analyte (“negative control”) or to contain an analyte (“positive control”). A positive control can comprise a known concentration of analyte.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time, but just over the time period being measured.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” are used interchangeably and refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effects of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached, for example, to an antibody or antigen to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Exemplary DR5 Agonists

Provided herein are methods of treating cancer comprising administering a DR5 agonist. Nonlimiting exemplary DR5 agonists include INBRX-109, eftozanermin alfa (ABBV-621), IGM-8444 (IGM Biosciences), BI 905711 (Boehringer Ingelheim), GEN1029 (HexaBody®-DR5/DR5; Genmab), TAS266 (Novartis), MM-201a (Merrimack Pharmaceuticals), MM201-b (Merrimack Pharmaceuticals), KMTR2 (Kyowa Hakko Kirin), DS-8273a (Daiichi Sankyo), dulanermin (Genentech/Amgen), lexatumumab (Human Genome Sciences/GSK), conatumumab (Amgen), drozitumab (Genentech/Roche), LBY135 (Novartis), and tigatuzumab (Daiichi Sankyo). In some embodiments, the DR5 agonist is a DR5-binding polypeptide. In some embodiments, a DR5-binding polypeptide provided herein is multivalent. In some embodiments, a DR5-binding polypeptide provided herein is at least tetravalent. In some embodiments, a DR5-binding polypeptide provided herein is tetravalent.

In various embodiments, a DR5-binding polypeptide comprises at least one VHH domain comprising CDR1 comprising the sequence of SEQ ID NO: 1, a CDR2 comprising the sequence of SEQ ID NO: 2, and a CDR3 comprising the sequence of SEQ ID NO: 3. In some embodiments, at least one VHH domain is humanized. In some embodiments, a DR5-binding polypeptide comprises at least one VHH domain comprising an amino acid sequence at least 90%, at least 95%, at least 99%, or 100% identical to the amino acid sequence of SEQ ID NO: 4. In some embodiments, a DR5-binding polypeptide comprises at least one VHH domain comprising the amino acid sequence of SEQ ID NO: 4.

In some embodiments, a DR5-binding polypeptide comprises at least one VHH domain that binds DR5 and an Fc region. In some embodiments, a DR5-binding polypeptide provided herein comprises two VHH domains that bind DR5 and an Fc region. In some embodiments, an Fc region mediates dimerization of the DR5-binding polypeptide at physiological conditions such that a dimer is formed that doubles the number of DR5 binding sites. For example, a DR5-binding polypeptide comprising two VHH domains that bind DR5 and an Fc region is divalent as a monomer, but at physiological conditions, the Fc region may mediate dimerization, such that the DR5-binding polypeptide is a tetravalent dimer under such conditions.

In various embodiments, a DR5-binding polypeptide is provided wherein each VHH domain comprises a CDR1 comprising the sequence of SEQ ID NO: 1, a CDR2 comprising the sequence of SEQ ID NO: 2, and a CDR3 comprising the sequence of SEQ ID NO: 3. In some embodiments, each VHH domain is humanized.

In some embodiments, a DR5-binding polypeptide comprises the structure VHH-linker-VHH-linker-Fc. In some embodiments, the VHH-linker-VHH portion of a DR5-binding polypeptide comprises an amino acid sequence at least 90%, at least 95%, at least 99%, or 100% identical to the amino acid sequence of SEQ ID NO: 5. In some embodiments, the VHH-linker-VHH portion of a DR5-binding polypeptide comprises the amino acid sequence of SEQ ID NO: 5. In some embodiments, the Fc comprises a hinge. In some such embodiments, the Fc comprises the amino acid sequence of SEQ ID NO: 6. In some embodiments, a DR5-binding polypeptide comprises an amino acid sequence at least 90%, at least 95%, at least 99%, or 100% identical to the amino acid sequence of SEQ ID NO: 7, which includes two VHH domains and an Fc region. In some embodiments, a DR5-binding polypeptide comprises the amino acid sequence of SEQ ID NO: 7, which includes two VHH domains and an Fc region. In some embodiments, a DR5-binding polypeptide consists of the amino acid sequence of SEQ ID NO: 7. A DR5-binding polypeptide consisting of the amino acid sequence of SEQ ID NO: 7, or SEQ ID NO: 7 lacking the terminal lysine, may be referred to as INBRX-109.

In some embodiments, a VHH domain that binds DR5 may be humanized. Humanized antibodies (such as sdAbs or VHH-containing polypeptides) are useful as therapeutic molecules because humanized antibodies reduce or eliminate the human immune response to non-human antibodies, which can result in an immune response to an antibody therapeutic, and decreased effectiveness of the therapeutic. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13:1619-1633, and are further described, for example, in Riechmann et al., (1988) Nature 332:323-329; Queen et al., (1989) Proc. Natl Acad. Sci. USA 86:10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272:10678-10684 and Rosok et al., (1996) J. Biol. Chem. 271:22611-22618). Typically, the FR regions of a VHH are replaced with human FR regions to make a humanized VHH. In some embodiments, certain FR residues of the human FR are replaced in order to improve one or more properties of the humanized VHH. VHH domains with such replaced residues are still referred to herein as “humanized.”

In various embodiments, an Fc region included in a DR5-binding polypeptide is a human Fc region, or is derived from a human Fc region.

In some embodiments, an Fc region included in a DR5-binding polypeptide is derived from a human Fc region, and comprises a three amino acid deletion in the lower hinge corresponding to IgG1 E233, L234, and L235, herein referred to as “Fc xELL.” Fc xELL polypeptides do not engage FcγRs and thus are referred to as “effector silent” or “effector null”, however in some embodiments, xELL Fc regions bind FcRn and therefore have extended half-life and transcytosis associated with FcRn mediated recycling. In some embodiments, the Fc region is a human IgG1 xELL Fc region.

### Exemplary IAP Antagonists

Provided herein are methods of treating cancer comprising administering an IAP antagonist. In some embodiments, the IAP antagonist is a small molecule. In some embodiments, the IAP antagonist is APG-1387 (Ascentage Pharma Group International), birinapant (IGM Biosciences, Inc.), AZD5582 (AstraZeneca), LCL161 (Novartis), Debio 1143 (Merck, Debiopharm), or ASTX660 (Astex Pharmaceuticals, Inc.).

In some embodiments, the IAP antagonist is APG-1387. APG-1387 (also known as SM-1387) is a bivalent IAP antagonist having the structure

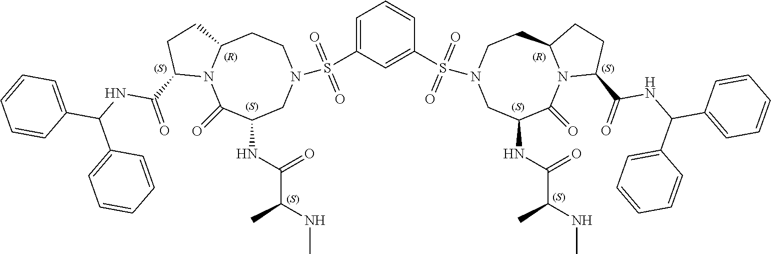

or a pharmaceutically acceptable salt or hydrate thereof. See, e.g., U.S. Pat. No. 8,883,771 and WO2014/031487. APG-1387 is a SMAC mimetic, and has potent in vitro and in vivo antitumor activity in solid malignancies.

In some embodiments, the IAP antagonist is birinapant. Birinapant (also known as TL32711) is a bivalent IAP antagonist having the structure:

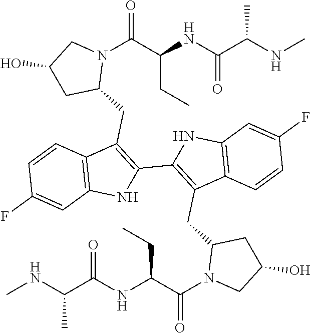

or a pharmaceutically acceptable salt or hydrate thereof. See, e.g., U.S. Pat. No. 8,283,372. Birinapant is a SMAC mimetic that binds to XIAP, CIAP1, and CIAP2, and has in vitro and in vivo antitumor activity in solid malignancies.

In some embodiments, the IAP antagonist is AZD5582. AZD5582 is a bivalent IAP antagonist having the structure:

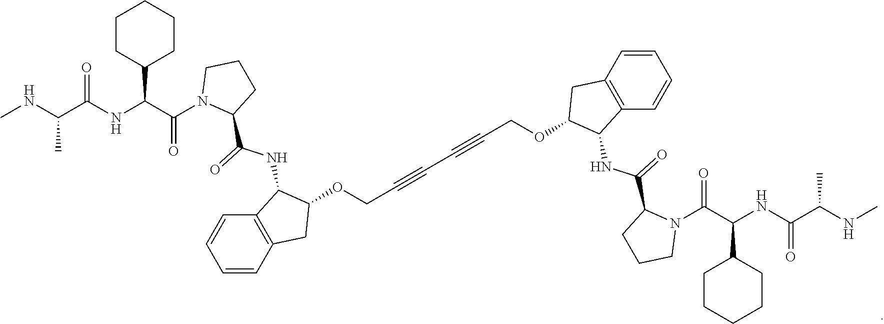

or a pharmaceutically acceptable salt or hydrate thereof. See, e.g., WO2010/142994A1. AZD5582 is a SMAC mimetic.

In some embodiments, the IAP antagonist is LCL161. LCL161 is a monovalent IAP antagonist having the structure:

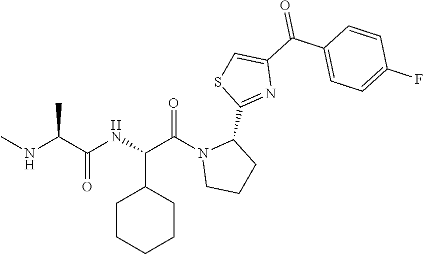 [SMILES: CN[C@@H](C)C(=O)N[C@H](C(=O)N1CCC[C@H]1c1nc(C(=O)c2ccc(F)cc2)cs1)C1CCCCC1]

or a pharmaceutically acceptable salt or hydrate thereof. See, e.g., WO 2011/019782. LCL161 is a SMAC mimetic that binds to XIAP, CIAP1, and CIAP2.

In some embodiments, the IAP antagonist is Debio 1143. Debio 1143 (also known as xevinapant, AT-406, SM-406, and ARRY-334543) is a monovalent IAP antagonist having the structure:

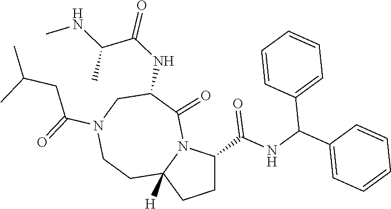 [SMILES: CN[C@@H](C)C(=O)N[C@H]1CN(C(=O)CC(C)C)CC[C@H]2CC[C@@H](C(=O)NC(c3ccccc3)c3ccccc3)N2C1=O]

or a pharmaceutically acceptable salt or hydrate thereof. See, e.g., PLOS ONE 14 (2): e0211746 (2019). Debio 1143 is a SMAC mimetic that binds to XIAP, CIAP1, and CIAP2.

In some embodiments, the IAP antagonist is ASTX660. ASTX660 (also known as tolinapant), is a monovalent IAP antagonist having the structure:

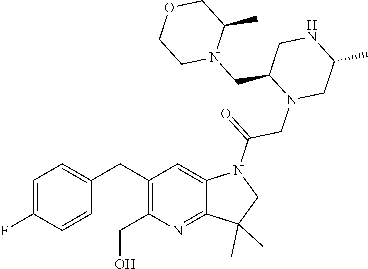 [SMILES: C[C@@H]1CN(CC(=O)N2CC(C)(C)c3nc(CO)c(Cc4ccc(F)cc4)cc32)[C@@H](CN2CCOC[C@H]2C)CN1]

or a pharmaceutically acceptable salt or hydrate thereof. See, e.g., J. Med. Chem., 61 (16): 7314-7329 (2018); U.S. Pat. No. 9,783,538; WO 2021/225955 A1. ASTX660 is a non-peptidomimetic that antagonizes XIAP, CIAP1, and CIAP2.

### Polypeptide Expression and Production

Nucleic acid molecules comprising polynucleotides that encode a DR5-binding polypeptide are provided. In some embodiments, the nucleic acid molecule may also encode a leader sequence that directs secretion of the DR5-binding polypeptide, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode the DR5-binding polypeptide described herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In some embodiments, a DR5-binding polypeptide may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells. In some embodiments, the DR5-binding polypeptide may be expressed in yeast. See, e.g., U.S. Publication No. US 2006/0270045 A1. In some embodiments, a particular eukaryotic host cell is selected based on its ability to make desired post-translational modifications to the polypeptide. For example, in some embodiments, CHO cells produce polypeptides that have a higher level of sialylation than the same polypeptide produced in 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses a DR5-binding polypeptide described herein is provided. The DR5-binding polypeptides expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify a DR5-binding polypeptide that comprises an Fc region. Hydrophobic interactive chromatography, for example, a butyl or phenyl column, may also be suitable for purifying some polypeptides such as antibodies. Ion exchange chromatography (for example anion exchange chromatography and/or cation exchange chromatography) may also be suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also be suitable for purifying some polypeptides such as antibodies. Many methods of purifying polypeptides are known in the art.

In some embodiments, the DR5-binding polypeptide is produced in a cell-free system. Nonlimiting exemplary cell-free systems are described, for example, in Sitaraman et al., Methods Mol. Biol. 498:229-44 (2009); Spirin, Trends Biotechnol. 22:538-45 (2004); Endo et al., Biotechnol. Adv. 21:695-713 (2003).

In some embodiments, a DR5-binding polypeptide prepared by the methods described above are provided. In some embodiments, the DR5-binding polypeptide is prepared in a host cell. In some embodiments, the DR5-binding polypeptide is prepared in a cell-free system. In some embodiments, the DR5-binding polypeptide is purified. In some embodiments, a cell culture media comprising a DR5-binding polypeptide is provided.

In some embodiments, compositions comprising antibodies prepared by the methods described above are provided. In some embodiments, the composition comprises a DR5-binding polypeptide prepared in a host cell. In some embodiments, the composition comprises a DR5-binding polypeptide prepared in a cell-free system. In some embodiments, the composition comprises a purified DR5-binding polypeptide.

### Pharmaceutical Compositions

In some embodiments, compositions comprising DR5 agonists and/or IAP antagonists are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available.

In some embodiments, INBRX-109 is provided in a formulation comprising 50 mg/mL INBRX-109, 10 mM histidine HCl, 8% w/v sucrose, 0.2% w/v poloxamer-88, pH 6.0.

### Exemplary Methods of Treating Cancer Using a DR5 Agonist and an IAP Antagonist

In some embodiments, methods of treating cancer in an individual comprising administering a DR5 agonist and an IAP antagonist are provided.

In some embodiments, the method comprises administering to the individual an effective amount of a DR5 agonist and an IAP antagonist. Such methods of treatment may be in humans or animals. In some embodiments, methods of treating humans are provided. Nonlimiting exemplary cancers that may be treated with the combination of a DR5 agonists and IAP antagonists provided herein include adrenal cancer; astrocytoma; basal cell carcinoma; biliary tract cancer; bladder cancer; bone cancer; brain and central nervous system cancer; breast cancer; cancer of the peritoneum; cervical cancer; choriocarcinoma; chondrosarcoma; Ewing sarcoma; colon and rectum cancer (colorectal cancer); connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; leukemia; liver cancer; lung cancer; small-cell lung cancer; non-small cell lung cancer; adenocarcinoma of the lung; squamous carcinoma of the lung; melanoma; myeloma; neuroblastoma; oral cavity cancer (lip, tongue, mouth, and pharynx); ovarian cancer; pancreatic cancer, such as pancreatic adenocarcinoma; pituitary gland cancer; prostate cancer; retinoblastoma; rhabdomyosarcoma; rectal cancer; cancer of the respiratory system; mesothelioma; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

The DR5 agonist and IAP antagonist can be administered as needed to subjects. Determination of the frequency of administration of each agent can be made by persons skilled in the art, such as an attending physician based on considerations of the condition being treated, age of the subject being treated, severity of the condition being treated, general state of health of the subject being treated and the like. In some embodiments, an effective dose of one or more therapeutic agents is administered to a subject one or more times. In some embodiments, an effective dose of a DR5 agonist and/or an IAP antagonist is administered to the subject daily, semiweekly, weekly, every two weeks, once a month, etc. An effective dose of a DR5 agonist and/or an IAP antagonist is administered to the subject at least once. In some embodiments, the effective dose of a DR5 agonist and/or an IAP antagonist may be administered multiple times, including multiple times over the course of at least a month, at least six months, or at least a year.

In some embodiments, a DR5 agonist is administered in an amount effective for treating (including prophylaxis of) cancer. The therapeutically effective amount is typically dependent on the weight of the subject being treated, his or her physical or health condition, the extensiveness of the condition to be treated, or the age of the subject being treated. In general, DR5-binding polypeptides may be administered in an amount in the range of about 0.05 mg/kg body weight to about 100 mg/kg body weight per dose, or in the range of about 10 μg/kg body weight to about 100 mg/kg body weight per dose, or in the range of about 50 μg/kg body weight to about 5 mg/kg body weight per dose, or in the range of about 100 μg/kg body weight to about 10 mg/kg body weight per dose, or in the range of about 100 μg/kg body weight to about 20 mg/kg body weight per dose, or in the range of about 0.5 mg/kg body weight to about 20 mg/kg body weight per dose, or in the range of about 1 mg/kg body weight to about 10 mg/kg body weight per dose.

In some embodiments, INBRX-109 is administered at a dose between about 1 mg/kg body weight to about 30 mg/kg body weight. In some embodiments, INBRX-109 is administered at a dose between about 1 mg/kg body weight to about 10 mg/kg body weight. In some embodiments, INBRX-109 is administered at a dose between about 1 mg/kg body weight to about 2 mg/kg body weight. In some embodiments, INBRX-109 is administered at a dose between about 2 mg/kg body weight to about 3 mg/kg body weight. In some embodiments, INBRX-109 is administered at a dose of about 3 mg/kg body weight. In some embodiments, INBRX-109 is administered once every 1 week, once every 2 weeks, once every 3 weeks, or once every 4 weeks. In some embodiments, INBRX-109 is administered once every 3 weeks.

In some embodiments, an IAP antagonist (or a pharmaceutically acceptable salt thereof or a hydrate) is administered at a dose of about 1 mg/m2 to about 1000 mg/m2, including, for example, about 10 mg/m2 to about 500 mg/m2, about 10 mg/m2 to about 300 mg/m2, or about 10 mg/m2 to about 200 mg/m2. In some embodiments, an IAP antagonist (or a pharmaceutically acceptable salt thereof or a hydrate) is administered at a dose of about 0.1 mg to about 10,000 mg, including, for example, 10 mg to 5,000 mg, or 10 mg to 1,000 mg, or 10 mg to 500 mg.

In some embodiments, APG-1387 is administered at a dose between about 0.3 mg and about 60 mg. In some embodiments, APG-1387 is administered once every 1 week. In some embodiments, birinapant is administered at a dose between about 2.8 and about 47 mg/m2. In some embodiments, LCL161 is administered at a dose between about 10 to about 3,000 mg. In some embodiments, LCL161 is administered at a dose of about 900 mg to about 1,800 mg. In some embodiments, LCL161 is administered weekly. In some embodiments, Debio 1143 is administered at a dose between about 5 and about 900 mg. In some embodiments, Debio 1143 is administered at a dose of about 100 mg to about 200 mg. In some embodiments, Debio 1143 is administered at a dose of about 200 mg. In some embodiments, Debio 1143 is administered daily. In some embodiments, ASTX660 is administered at a dose between about 15 mg and about 270 mg. In some embodiments, ASTX660 is administered daily, optionally on a 7-day-on/7-day-off schedule.

In some embodiments, a therapeutic agent can be administered in vivo by various routes, including, but not limited to, oral, intramuscular, intravenous, intra-arterial, parenteral, intraperitoneal, or subcutaneous. The appropriate formulation and route of administration may be selected according to the intended application.

In some embodiments, a DR5 agonist and an IAP antagonist are administered separately. In some embodiments, a DR5 agonist and an IAP antagonist are administered sequentially. In some embodiments, at least one dose of DR5 agonist is administered before an IAP antagonist. In some embodiments, at least one dose of DR5 agonist is administered after an IAP antagonist.

In some embodiments, a DR5 agonist and an IAP antagonist are administered concurrently.

In some embodiments, a DR5 agonist and an IAP antagonist act synergistically.

In some embodiments, synergy is determined in an in vitro cell survival assay. In some embodiments, administration of the DR5 agonist and the IAP antagonist results in a synergistic effect, compared to each agent administered alone.

In some embodiments, a DR5 agonist is provided for use in a method of treating cancer, wherein the method comprises administering the DR5 agonist in combination with an IAP antagonist.

In some embodiments, use of a DR5 agonist for the manufacture of a medicament for treating cancer is provided, wherein the medicament is for administration with an IAP antagonist.

### Kits

Also provided are articles of manufacture and kits that include any of the DR5 agonists and/or IAP antagonists provided herein and suitable packaging. In some embodiments, the invention includes a kit with (i) a formulation comprising a DR5 agonist, (ii) a formulation comprising an IAP antagonist, and (iii) instructions for using the kit to administer the formulations to an individual. In some embodiments, the invention includes a kit with (i) a formulation comprising a DR5 agonist, and (ii) instructions for using the kit to administer the formulations to an individual in combination with an IAP antagonist. In some embodiments, the invention includes a kit with (i) a formulation comprising an IAP antagonist, and (ii) instructions for using the kit to administer the formulations to an individual in combination with a DR5 agonist.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the DR5 agonists and/or IAP antagonists generally include information as to dosage, dosing schedule, and route of administration for the intended treatment or industrial use. The kit may further comprise a description of selecting an individual suitable or treatment.

The containers may be unit doses, bulk packages (e.g., multi-dose packages) or sub-unit doses. For example, kits may also be provided that contain sufficient dosages of molecules disclosed herein to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of molecules and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous solution of DR5 agonist.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Combinatorial Activity of INBRX-109 and Various IAP Antagonists

The combination of INBRX-109 and various IAP antagonists was tested on various cancer cell lines to determine cytotoxicity to cancer cells.

### Assay Protocol

Day 1-Cell Seeding. Monolayer cultures of each cell line were harvested for compound screening as detailed below. Culture medium was aspirated, and the cells were washed once with PBS. Accutase was added and flasks were incubated at 37° C. until cells became detached. An equal volume of complete medium was added to quench the Accutase, and the cells were then pipetted up and down several times to generate a homogenous single cell suspension. The density and viability of cells was determined by Trypan Blue using a TC20 Automated Cell Counter. Experimental cells were resuspended to a concentration of 0.17×10\hat{ }6/mL in EMEM/10% FBS/Anti-Anti media (complete EMEM) and seeded in the inner wells of 384-well luminescence plates at 15 μL/well (2,500/well final). Each cell line was plated in duplicate on separate plates. Outer wells were filled with 50 μL PBS, then plates were incubated in a humidified temperature-controlled 37° C. tissue culture incubator at 5% CO2 overnight for 16 hours.

Test Article Preparation for IAP Antagonists: All small molecules were purchased as 10 mM solutions in DMSO from MedChemExpress. These test articles were then aliquoted and stored at −80° C. Aliquots were thawed immediately prior to dilution and use in the assay. A 500× master plate of serial dilutions (6-point 2-fold dilutions in 100% DMSO, plus a DMSO only control) were prepared and mixed gently with a pipette. To generate a 5× working dilution plate of the small molecules, a 1:100 dilution was performed from the 500× plate into complete media (EMEM).

Test Article Preparation for INBRX-109. INBRX-109 assay concentration ranges were chosen to bracket the minimal and maximal activity seen in previous cytotoxicity assays with several cancerous cell lines, with 1 nM defined as the maximally efficacious concentration. A 50× master plate of INBRX-109 serial dilutions (6-point 10-fold dilutions in complete EMEM starting at 500 nM, plus a complete EMEM only control) was prepared and mixed gently with pipette. To generate a 5× working dilution plate of INBRX-109, each well from the 50× master plate was diluted 1:10 into complete EMEM.

Day 2—Test and Control Article Addition. IAP antagonist small molecule working dilutions (5 μL of 5×), INBRX-109 working dilutions (5 μL of 5×), or Staurosporine positive control (5 μL of 5×) were added to the respective experimental wells. IAP antagonist titrations were performed going across the plate, and INBRX-109 titrations were done going down the plate, leading to a matrix of all possible combinations of the two test articles. These test articles were added, in duplicate, to each cell line. The plates were then centrifuged at 400× g for 1 minute, then incubated in a 37° C. humidity-controlled tissue culture incubator (5% CO2) for 48 hours.

Day 4—Viability Measurements. Plates were equilibrated to room temperature for 10 minutes, then 25 μL of CellTiter-Glo 2.0® was added to each well. Plates were spun for 1 minute at 400×g, then covered and incubated in the dark for 10 minutes at room temperature. Any visible bubbles were removed with 100% ethanol vapor, then luminescence (RLU) was read on the Spectra Max M5e plate reader, using the 384-well opaque plate setting and SoftMaxPro v5.4 software with an integration time of 50 milliseconds. To determine the effect of test articles on cell viability, raw RLU values were exported to Excel and percent survival was calculated as a percentage of vehicle control (0.2% DMSO in EMEM), where the vehicle control is set at 100%. Data was graphed in GraphPad Prism 7.

### Results

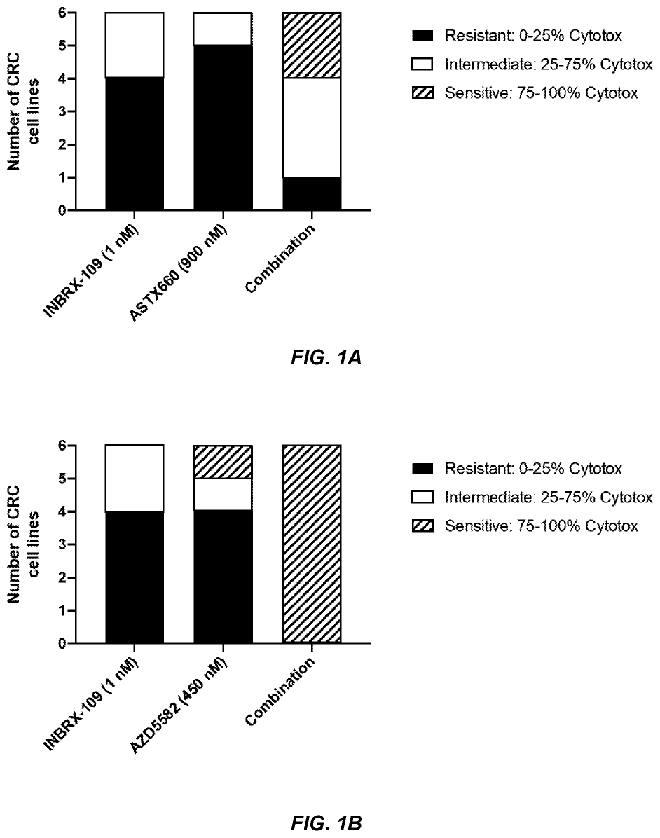

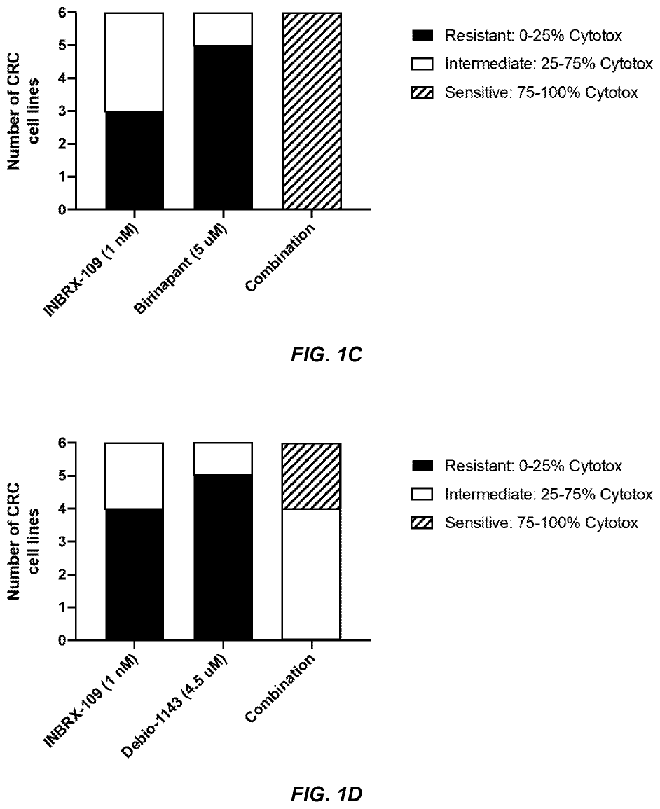

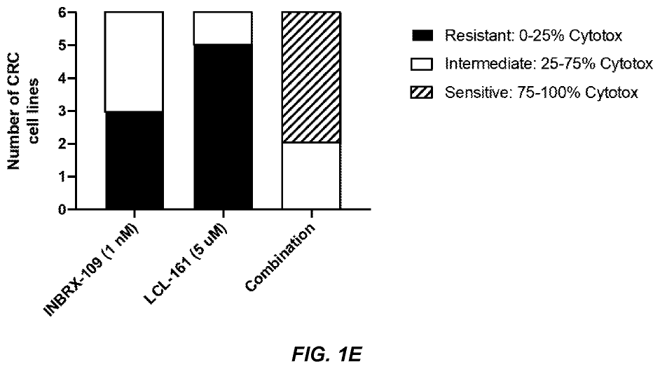

FIG. 1A-1E shows results of experiments in which six different cancer cell lines were contacted with INBRX-109 (1 nM), an IAP antagonist, or a combination of INBRX-109 (1 nM) and an IAP antagonist. The bar graphs show the number of cell lines (out of six tested) that are resistant (0-25% cytotoxicity), intermediate (25-75% cytotoxicity), or sensitive (75-100% cytotoxicity). The six cell lines tested were: LOVO, LS174T, SW620, SNU-C2B, HT-29, and LS411N. The number of cancer cell lines is shown on the y-axis of each bar graph. The results are shown for IAP antagonists ASTX660 (900 nM) (FIG. 1A), AZD5582 (450 nM) (FIG. 1B), birinapant (5 μM) (FIG. 1C), Debio 1143 (4.5 μM) (FIG. 1D), and LCL-161 (5 μM) (FIG. 1E).

These results are also shown in Tables 2-6. Percent cytotoxicity was calculated by subtracting the % survival from 100.

TABLE 2

| | % cytotoxicity | | |
|---|---|---|---|
| | INBRX-109 | ASTX660 | INBRX-109 + |
| Cell line | (1 nM) | (900 nM) | ASTX660 |
| LOVO | 7.0 | 6.5 | 11.9 |
| LS174T | 24.7 | n.d. | 61.8 |
| SW620 | 11.6 | 2.6 | 42.4 |
| SNU-C2B | 26.7 | 31.1 | 62.6 |
| HT-29 | 35.9 | 0.3 | 87.5 |
| LS411N | 9.7 | 5.9 | 75.8 |

TABLE 3

| | % cytotoxicity | | |
|---|---|---|---|
| | INBRX-109 | AZD5582 | INBRX-109 + |
| Cell line | (1 nM) | (450 nM) | AZD5582 |
| LOVO | 15.1 | 28.2 | 88.4 |
| LS174T | 24.7 | n.d. | 90.8 |
| SW620 | 22.0 | 4.1 | 93.0 |
| SNU-C2B | 40.4 | 75.3 | 75.2 |
| HT-29 | 27.1 | 23.4 | 99.5 |
| LS411N | 15.1 | 12.7 | 94.9 |

TABLE 4

| | % cytotoxicity | | |
|---|---|---|---|
| | INBRX-109 | Birinapant | INBRX-109 + |
| Cell line | (1 nM) | (5 uM) | Birinapant |
| LOVO | 15.1 | n.d. | 77.0 |
| LS174T | 28.8 | n.d. | 90.4 |
| SW620 | 17.0 | 11.7 | 90.7 |
| SNU-C2B | 30.4 | 71.1 | 75.9 |
| HT-29 | 30.1 | 20.4 | 98.6 |
| LS411N | 7.5 | 14.3 | 93.8 |

TABLE 5

| | % cytotoxicity | | |
|---|---|---|---|
| | INBRX-109 | Debio-1143 | INBRX-109 + |
| Cell line | (1 nM) | (4.5 uM) | Debio-1143 |
| LOVO | 11.8 | n.d. | 60.2 |
| LS174T | 33.9 | n.d. | 69.0 |
| SW620 | 15.7 | 5.5 | 51.1 |
| SNU-C2B | 17.4 | 44.8 | 69.7 |
| HT-29 | 26.3 | n.d. | 93.1 |
| LS411N | 18.3 | 0.4 | 86.5 |

TABLE 6

| | % cytotoxicity | | |
|---|---|---|---|
| | INBRX-109 | LCL-161 | INBRX-109 + |
| Cell line | (1 nM) | (5 uM) | LCL-161 |
| LOVO | n.d. | n.d. | 89.3 |
| LS174T | 27.6 | 9.0 | 90.7 |
| SW620 | 10.8 | 4.7 | 68.9 |
| SNU-C2B | 28.3 | 72.9 | 71.6 |
| HT-29 | 36.1 | 6.2 | 98.8 |
| LS411N | 11.7 | 10.5 | 93.0 |

The combination of INBRX-109 and an IAP antagonist showed increased cell killing on almost all cancer cell lines tested in this screen, and for every IAP antagonist, compared to either INBRX-109 or an IAP antagonist alone.

This data suggests that the combination of a DR5 agonist, such as INBRX-109, and an IAP antagonist, such as ASTX660, AZD5582, birinapant, Debio 1143, or LCL-161, results in improved or synergistic cancer cell killing compared the individual drugs themselves.

### Example 2: Combinatorial Activity of INBRX-109 and LCL-161, or INBRX-109 and Birinapant

The combination of INBRX-109 and IAP antagonist LCL-161 or birinapant was tested on the LS174T and SW620 cancer cell lines to determine cytotoxicity to cancer cells, using a similar assay as described in Example 1.

### Results

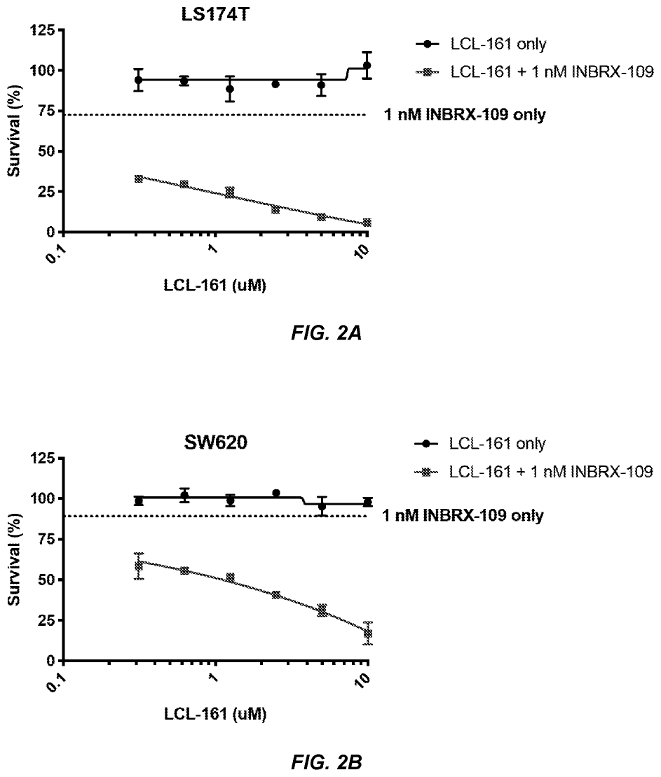

FIG. 2A-2B shows results of an LCL-161 titration experiment in which cancer cell lines were contacted with different concentrations of LCL-161, alone or in combination with 1 nM INBRX-109. Percent survival of cancer cells is shown on the y-axis of each graph. The results are shown for cancer cell lines LS174T (FIG. 2A) and SW620 (FIG. 2B). The dotted line labeled “1 nM INBRX-109 only” shows the percent survival of cancer cells treated with 1 nM INBRX-109 alone.

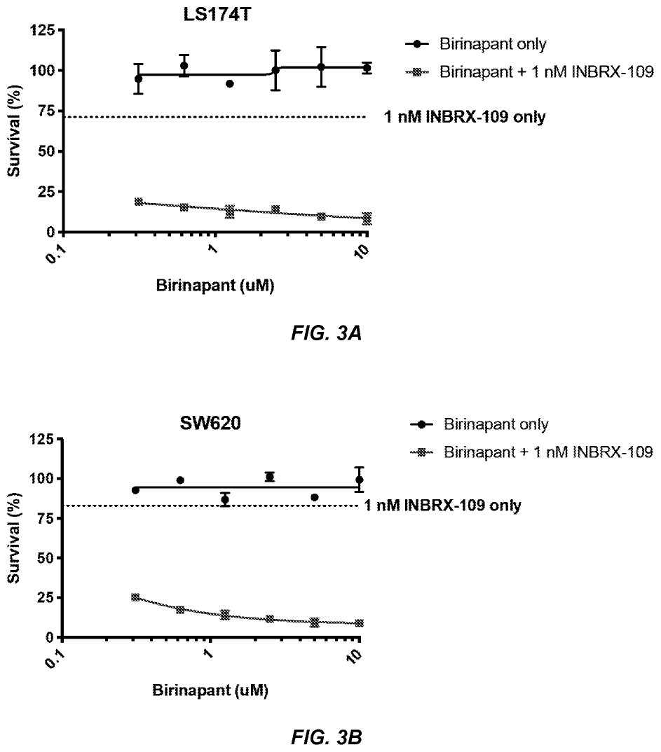

FIG. 3A-3B shows results of a birinapant titration experiment in which cancer cell lines were contacted with different concentrations of birinapant, alone or in combination with 1 nM INBRX-109. Percent survival of cancer cells is shown on the y-axis of each graph. The results are shown for cancer cell lines LS174T (FIG. 3A) and SW620 (FIG. 3B). The dotted line labeled “1 nM INBRX-109 only” shows the percent survival of cancer cells treated with 1 nM INBRX-109 alone.

The combination of INBRX-109 and either LCL-161 or birinapant showed increased cell killing on the cancer cell lines tested in this screen, compared to either agent alone.

This data suggests that the combination of a DR5 agonist, such as INBRX-109, and an IAP antagonist, such as LCL-161 or birinapant, results in improved or synergistic cancer cell killing compared the individual drugs themselves.

### Example 3: Combinatorial Activity of INBRX-109 and Debio-1143 on Additional Cancer Cell Lines

The combination of INBRX-109 and Debio-1143 was tested on various head and neck cancer cell lines to determine cytotoxicity to cancer cells.

### Assay Protocol

Day 1—Cell Seeding. Monolayer cultures of each cancer cell line (see Table 7) were harvested during the logarithmic growth period. The density and viability of cells was determined by Trypan Blue using a Vi cell counter, cells were diluted in the appropriate growth media (see Table 7) and 90 μL cell suspensions were added to 96-well plates for compound screening as detailed below. Two duplicate plates were set up for day 0 reading (T0) and for reading at the end point (T48).

TABLE 7

| Cell line | | |
|---|---|---|
| name | Tissue Origin | Cell Culture Medium |
| COLO-680N | Head & Neck/Esophagus | RPMI-1640 + 10% FBS |
| KYSE-150 | Head & Neck/Esophagus | RPMI1640/F12 + 2% h.i.FBS |
| KYSE-270 | Head & Neck/Esophagus | RPMI1640/F12 + 2% h.i.FBS |
| KYSE-410 | Head & Neck/Esophagus | RPMI1640 + 5~10% FBS |
| KYSE-70 | Head & Neck/Esophagus | RPMI1640 + 10% h.i.FBS |
| T.Tn | Head & Neck/Esophagus | DMEM:Ham's F-12 = 1:1 + |
| | | 10% FBS |
| TE-1 | Esophagus | RPMI1640 + 10% FBS |
| RPMI-2650 | Head & Neck/Nose | (MEM + 0.01 mM NEAA) + |
| | | 10% FBS |
| Detroit 562 | Pharynx | (MEM + 0.01 mM NEAA) + |
| | | 10% FBS |
| FaDu | Pharynx | (MEM + 0.01 mM NEAA) + |
| | | 10% FBS |
| A253 | submaxillary salivary | Mc'oy's 5a + 10% FBS |
| | gland | |
| SW-579 | Head & Neck/Thyroid | L-15(100% air) + 10% FBS |
| CAL-27 | Head & Neck/Tongue | DMEM + 10% FBS |
| OSC-19 | Head & Neck/Tongue | DMEM/F12 + 10% FBS |
| SCC-15 | Head & Neck | DMEM/F12 + 10% FBS + |
| | | 400 ng/ml Hydrocortisone |
| SCC-25 | Head & Neck/Tongue | DMEM/F12 + 10% FBS + |
| | | 400 ng/ml Hydrocortisone |
| SCC-4 | Head & Neck/Tongue | DMEM/F12 + 10% FBS + |
| | | 400 ng/ml Hydrocortisone |
| SCC-9 | Head & Neck/Tongue | DMEM/F12 + 10% FBS + |
| | | 400 ng/ml Hydrocortisone |

Test Article Preparation: A 1000× master plate of serial dilutions (9-point 2-fold dilutions of Debio-1143 in 100% DMSO starting at 9000 μM, plus a DMSO only control) were prepared and mixed gently with a pipette. To generate a 10× working dilution plate of the small molecules, a 1:100 dilution was performed from the 1000× plate into the appropriate media. A 10× stock of INBRX-109 (10 nM) was prepared.

Day 2—Compound Treatment. 10 μL (10×) from the 10× working plate and/or 10 μL (10×) INBRX-109 (final concentration 1 nM) was added to the respective experimental wells in triplicate. The plates were then incubated in a 37° C. humidity-controlled tissue culture incubator (5% CO2).

20 μL culture medium containing 1% DMSO and 50 μL CellTiter-Glo® Reagent was added to each well of the TO plates and the contents mixed, plate was incubated at room temperature for 30 min, and read on an En Vision Multi Label Reader.

Day 4—Viability Measurements. 50 μL CellTiter-Glo® Reagent was added to each well of the T48 plates and the contents mixed, plate was incubated at room temperature for 30 min, and read on an EnVision Multi Label Reader. The formula for calculating percent survival is: % survival=(Lum_{Test article}−Lum_{Medium control})/(Lum_{None treated}−Lum_{Medium control})×100%. Percent cytotoxicity was calculated by subtracting the % survival from 100.

### Results

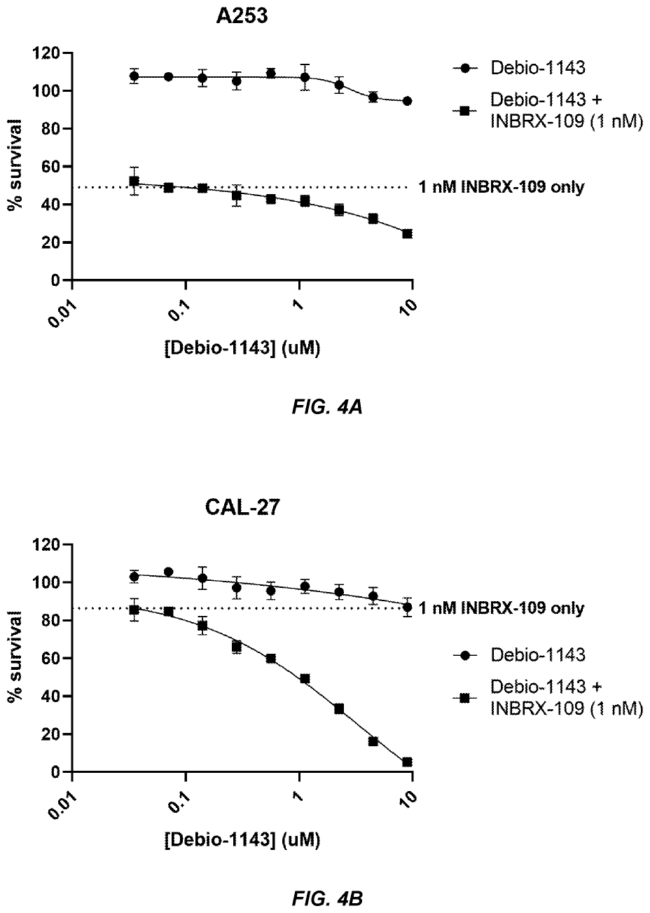

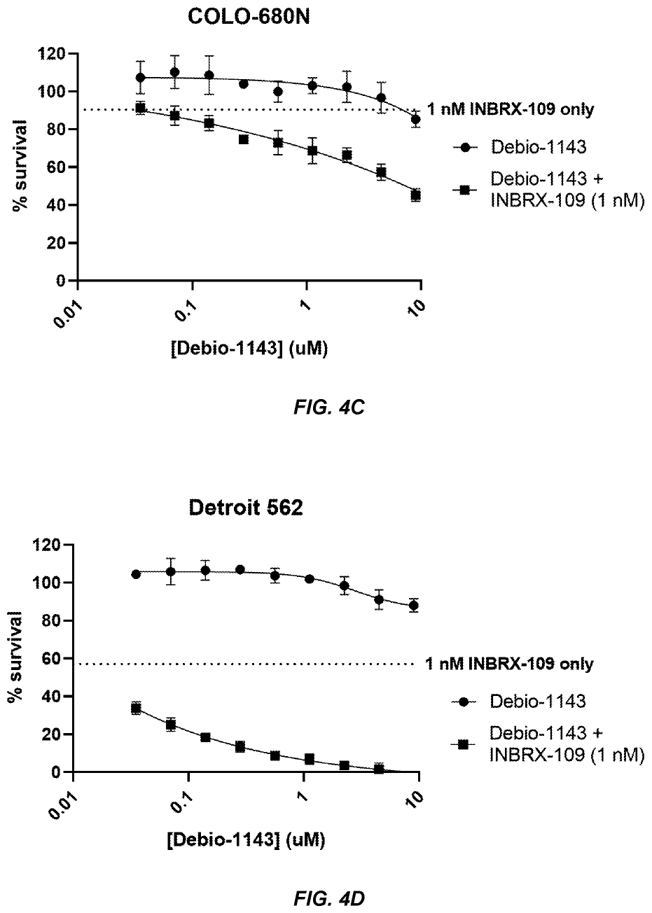

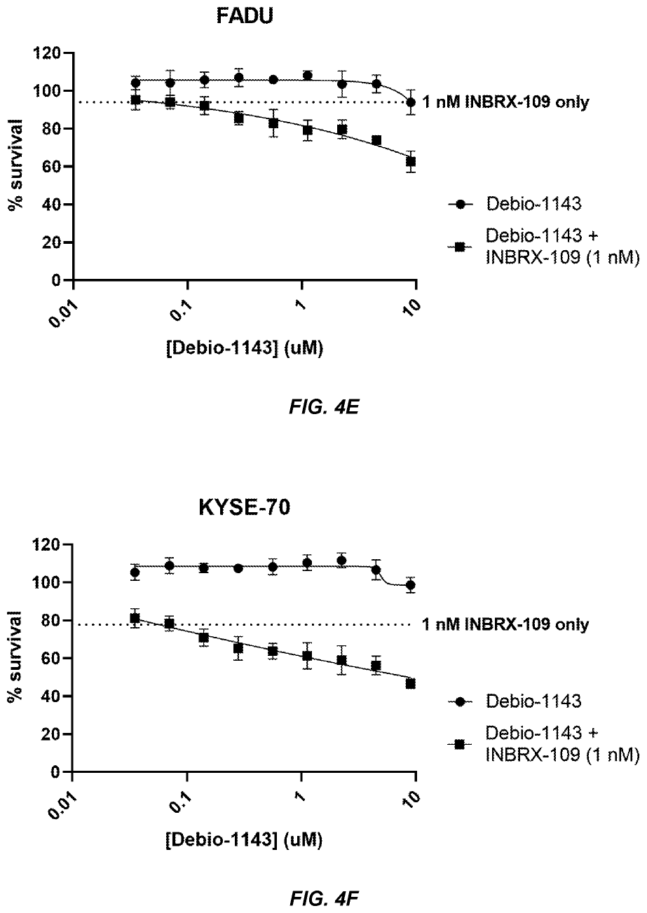

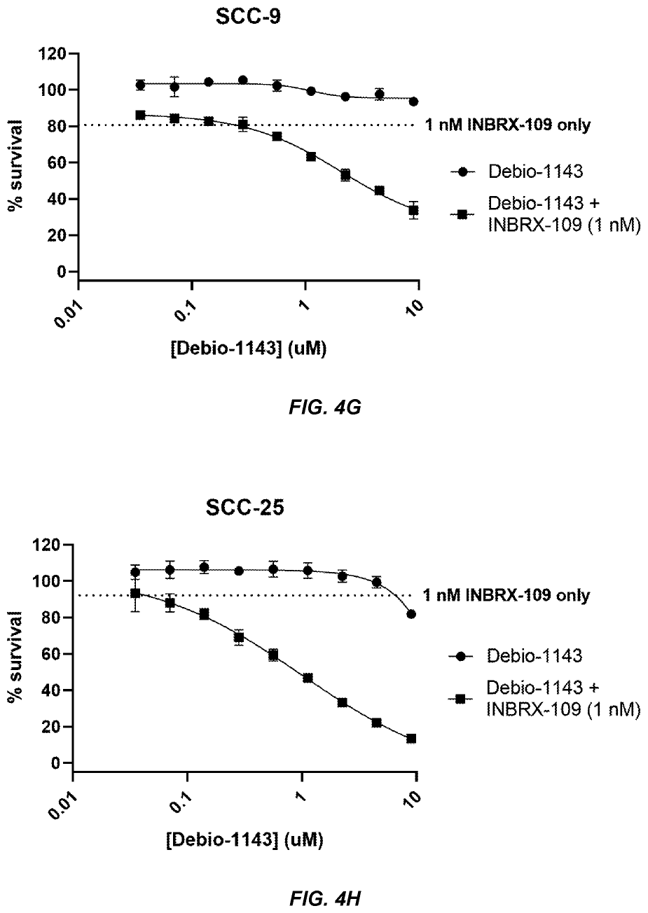

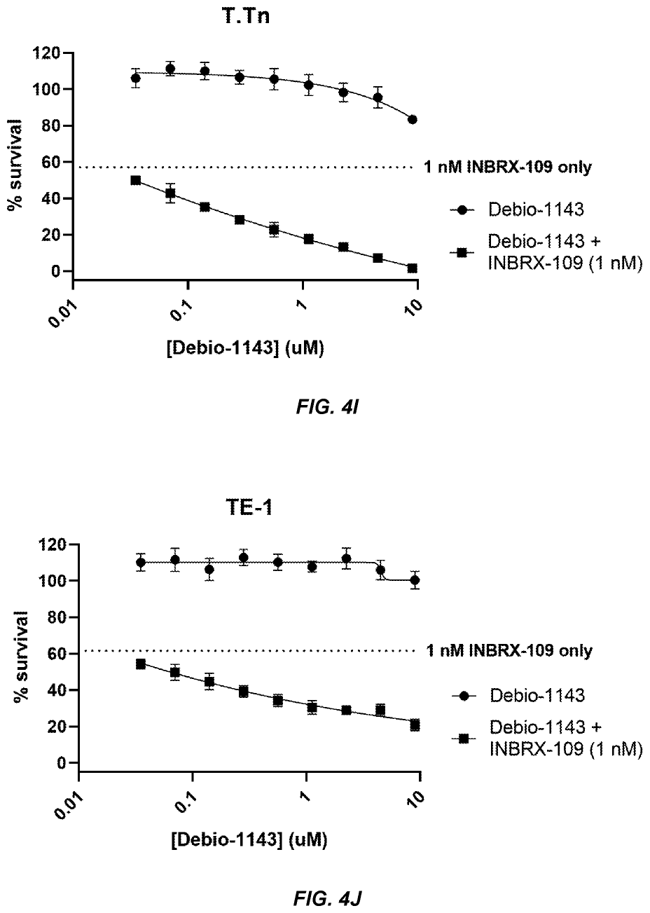

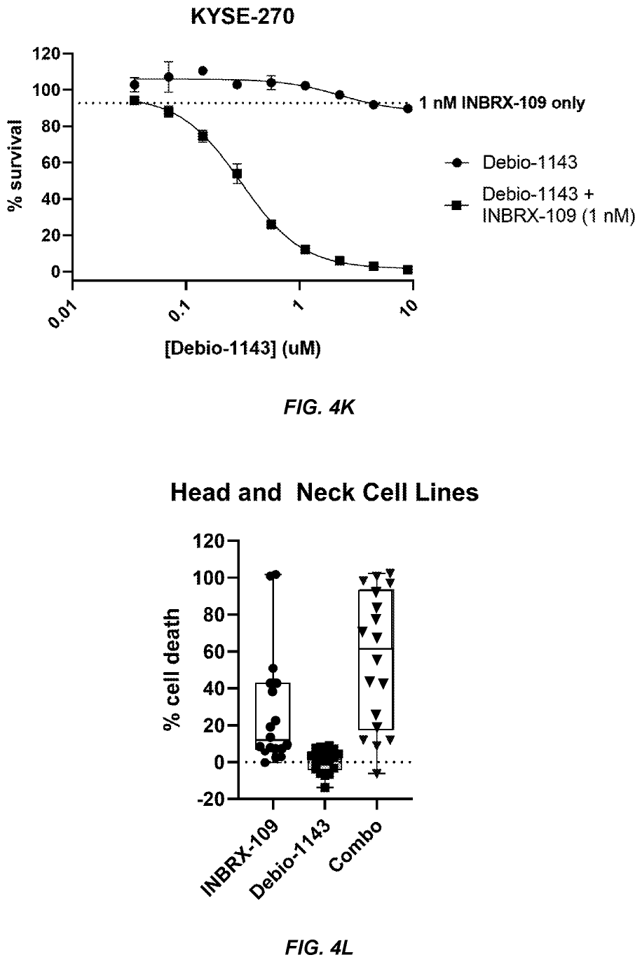

Table 8 presents the percent cytotoxicity for each cell line tested. FIG. 4A-4L shows results of a Debio-1143 titration experiment in which cancer cell lines were contacted with different concentrations of Debio-1143, alone or in combination with 1 nM INBRX-109. FIG. 4A-4K show the results for cancer cell lines that exhibited less than 90% killing with INBRX-109 alone and at least 25% maximum toxicity upon treatment with the combination: A253 (FIG. 4A), CAL-27 (FIG. 4B), COLO-680N (FIG. 4C), Detroit 562 (FIG. 4D), FADU (FIG. 4E), KYSE-70 (FIG. 4F), SCC-9 (FIG. 4G), SCC-25 (FIG. 4H), T.Tn (FIG. 4I), TE-1 (FIG. 4J), KYSE-270 (FIG. 4K). Percent survival of cancer cells is shown on the y-axis of each graph, and the dotted line labeled “1 nM INBRX-109 only” shows the percent survival of cancer cells treated with 1 nM INBRX-109 alone. The results shown in Table 8 are summarized in FIG. 4L, which plots % cytotoxicity (% cell death) for all 18 cell lines treated with INBRX-109 or Debio-1143 alone, or INBRX-109 and Debio-1143 in combination.

TABLE 8

| | % cytotoxicity | | |
|---|---|---|---|
| | INBRX-109 | Debio-1143 | Debio-1143 + |
| Cell Line | (1 nM) | (4.5 μM) | INBRX-109 |
| A253 | 50.9 | 3.2 | 67.5 |
| CAL-27 | 13.7 | 7.2 | 83.8 |
| COLO-680N | 9.6 | 3.4 | 42.7 |
| Detroit 562 | 42.9 | 9.0 | 98.3 |
| FADU | 6.1 | −3.7 | 25.9 |
| KYSE-410 | 2.6 | −3.3 | 19.1 |
| KYSE-70 | 22.3 | −6.7 | 43.7 |
| KYSE-150 | −0.3 | −13.7 | −6.2 |
| OSC-19 | 100.9 | −7.1 | 100.8 |
| SCC-25 | 7.9 | 0.6 | 77.8 |
| T.Tn | 42.9 | 4.4 | 92.7 |
| TE-1 | 38.4 | −6.0 | 71.0 |
| KYSE-270 | 7.3 | 8.2 | 96.9 |
| RPMI-2650 | 2.9 | 5.6 | 11.9 |
| SCC-4 | 8.5 | 0.7 | 8.9 |
| SCC-9 | 19.4 | 2.3 | 55.4 |
| SCC-15 | 7.3 | 0.9 | 12.1 |
| SW579 | 101.7 | 7.7 | 102.3 |

The combination of INBRX-109 and Debio-1143 showed increased cell killing on almost all cancer cell lines tested in this screen, compared to either INBRX-109 or an IAP antagonist alone. This data further suggests that the combination of a DR5 agonist, such as INBRX-109, and an IAP antagonist, such as Debio 1143, results in improved or synergistic cancer cell killing compared the individual drugs themselves.

### Example 4: Combinatorial Activity of INBRX-109 and Birinapant on Additional Cancer Cell Lines

The combination of INBRX-109 and birinapant was tested on various breast cancer cell lines (BT-20, BT-549, CAL-120, CAL-148, CAL-51, DU4475, HCC1143, HCC1187, HCC1395, HCC1806, HCC1937, HCC38, HCC70, HDQ-P1, Hs 578T, MX-1, and SUM159PT) to determine cytotoxicity to cancer cells, using a similar assay as described in Example 3. In additional studies, the combination of INBRX-109 and birinapant was tested on various chondrosarcoma (CAL-78, OUMS-27, SW1353, and H-EMC-SS), gastric (NCI-N87, SNU-5, and SNU-1), pancreatic (AsPC-1, Panc-1, MIA-PaCa-2, and SW1990), and lung (NCI-H460, HCC2935, NCI-H23, NCI-H2452, and A549) cancer cell lines, and the combination of INBRX-109 and Debio 1143 was tested on gastric (NCI-N87, SNU-5, and SNU-1), pancreatic (AsPC-1, Panc-1, MIA-PaCa-2, and SW1990), and lung (NCI-H460, HCC2935, NCI-H23, NCI-H2452, and A549) cancer cell lines to determine cytotoxicity to cancer cells, using a similar assay as described in Example 1.

### Results

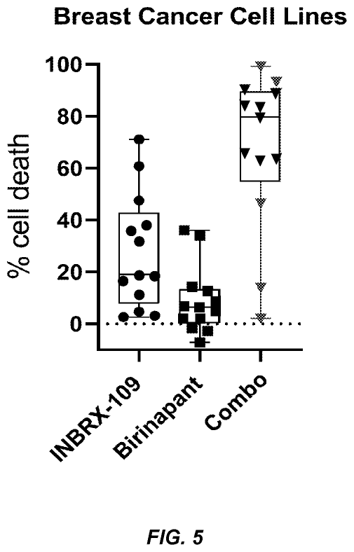

Table 9 presents the percent cytotoxicity for each breast cancer cell line tested. The results of Table 9 are summarized in FIG. 5, which plots % cytotoxicity (% cell death) for all 13 breast cancer cell lines treated with INBRX-109 or birinapant alone, or in combination.

TABLE 9

| | % cytotoxicity | | |
|---|---|---|---|
| | INBRX-109 | Birinapant | Birinapant + |
| Cell Line | (1 nM) | (3.16 μM) | INBRX-109 |
| CAL-120 | 60.78 | 2.75 | 90.40 |
| MX | 18.51 | −7.11 | 46.55 |
| BT-20 | 47.52 | 6.51 | 65.85 |
| BT-549 | 35.75 | 6.85 | 89.05 |
| HCC1937 | 3.16 | −2.77 | 2.09 |
| SUM159PT | 71.11 | −1.70 | 99.23 |
| HCC70 | 2.60 | 34.00 | 63.00 |
| HCC1806 | 11.34 | 5.17 | 79.54 |
| HCC1187 | 16.50 | 36.02 | 64.05 |
| DU4475 | 38.24 | 14.33 | 93.27 |
| CAL-51 | 4.70 | 2.35 | 13.99 |
| HCC1395 | 32.12 | 8.93 | 84.55 |
| HS 578T | 19.03 | 12.72 | 83.57 |

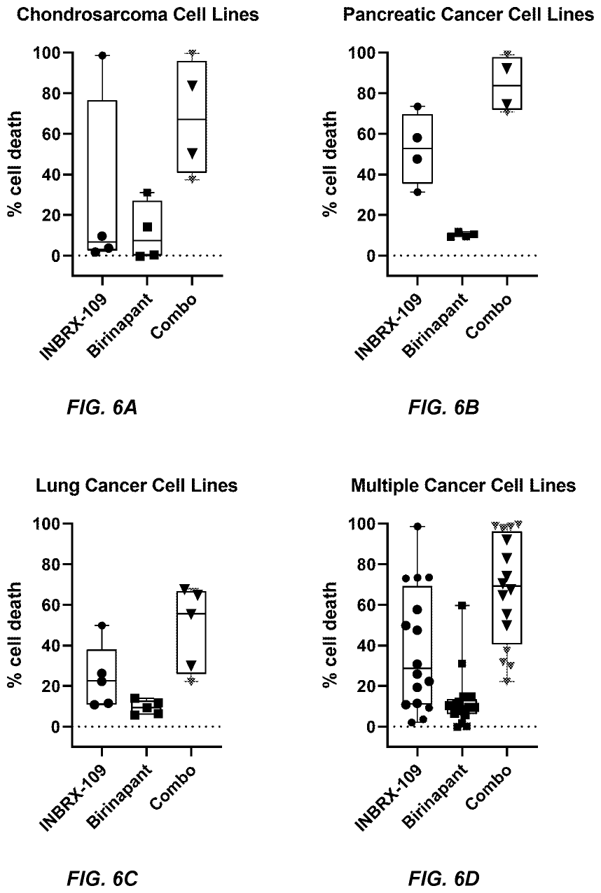

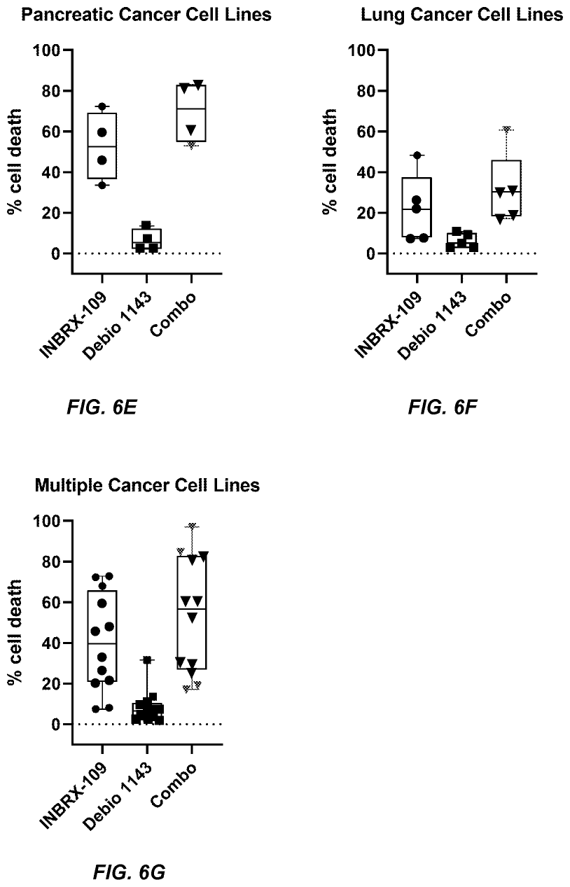

Table 10A presents the percent cytotoxicity for the chondrosarcoma, gastric, pancreatic, and lung cancer cell lines treated with INBRX-109 or birinapant alone, or in combination. Table 10B presents the percent cytotoxicity for the gastric, pancreatic, and lung cancer cell lines treated with INBRX-109 or Debio 1143 alone, or in combination. The results of Table 10A are summarized in FIG. 6A-6D, which plots % cytotoxicity (% cell death) for the cell lines treated with INBRX-109 or birinapant alone, or in combination. The results for the chondrosarcoma, pancreatic, and lung cancer cell lines are plotted in FIG. 6A-6C, respectively, and all the cell lines presented in Table 10A are plotted in FIG. 6D. The results of Table 10B are summarized in FIG. 6E-6G, which plots % cytotoxicity (% cell death) for the cell lines treated with INBRX-109 or Debio 1143 alone, or in combination. The results for the pancreatic, and lung cancer cell lines are plotted in FIGS. 6E and 6F, respectively, and all the cell lines presented in Table 10B are plotted in FIG. 6G.

TABLE 10A

| | % cytotoxicity | | |
|---|---|---|---|
| | INBRX-109 | Birinapant | Birinapant + |
| Cell Line | (1 nM) | (2 μM) | INBRX-109 |
| CAL-78 | 9.25 | 14.00 | 83.78 |
| OUMS-27 | 2.09 | −0.15 | 50.49 |
| SW1353 | 3.64 | 0.10 | 37.40 |
| H-EMC-SS | 98.61 | 31.05 | 99.58 |
| NCI-N87 | 19.92 | 1.82 | 31.83 |
| SNU-5 | 73.01 | 59.62 | 97.34 |
| SNU-1 | 73.34 | 7.62 | 98.51 |
| AsPC-1 | 57.92 | 9.46 | 74.48 |
| Panc-1 | 31.34 | 11.73 | 98.94 |
| MIA-PaCa-2 | 73.43 | 9.17 | 92.71 |
| SW1990 | 47.56 | 10.45 | 70.77 |
| NCI-H460 | 26.24 | 6.55 | 55.77 |
| HCC2935 | 22.42 | 13.93 | 65.37 |
| NCI-H23 | 11.52 | 9.30 | 22.14 |
| NCI-H2452 | 49.85 | 6.11 | 67.98 |
| A549 | 10.73 | 11.30 | 29.92 |

TABLE 10B

| | % cytotoxicity | | |
|---|---|---|---|
| | INBRX-109 | Debio 1143 | Debio 1143 + |
| Cell Line | (1 nM) | (2 μM) | INBRX-109 |
| NCI-N87 | 20.67 | 7.01 | 25.98 |
| SNU-5 | 67.97 | 31.53 | 84.74 |
| SNU-1 | 72.84 | 6.83 | 97.06 |
| AsPC-1 | 59.8 | 2.71 | 60.51 |
| Panc-1 | 33.54 | 13.61 | 81.57 |
| MIA-PaCa-2 | 72.23 | 7.37 | 83.09 |
| SW1990 | 45.71 | 2.23 | 52.92 |
| NCI-H460 | 26.51 | 3.43 | 30.21 |
| HCC2935 | 21.86 | 4.95 | 30.94 |
| NCI-H23 | 7.47 | 10.75 | 19.15 |
| NCI-H2452 | 48.21 | 2.82 | 60.63 |
| A549 | 8.1 | 9.61 | 17.17 |

The combination of INBRX-109 and the IAP antagonist (birinapant or Debio 1143) showed increased cell killing on almost all cancer cell lines tested in these screens, compared to either INBRX-109 or an IAP antagonist alone. This data further suggests that the combination of a DR5 agonist, such as INBRX-109, and an IAP antagonist, such as birinapant or Debio 1143, results in improved or synergistic cancer cell killing compared the individual drugs themselves.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

Table of Certain Sequences

| SEQ ID | | |
|---|---|---|
| NO | Description | Sequence |
| 1 | INBRX-109 | SGLTFPNYGM |
| | CDR1 | |
| 2 | INBRX-109 | AIYWSGGTVY |
| | CDR2 | |
| 3 | INBRX-109 | AVTIRGAATQTWKYDYW |
| | CDR3 | |
| 4 | INBRX-109 | EVQLLESGGGEVQPGGSLRLSCAASGLTFPNYGMGWFRQAPGKEREFVSAIYWS |
| | VHH | GGTVYYAESVKGRFTISRDNAKNTLYLQMSSLRAEDTAVYYCAVTIRGAATQTW |
| | | KYDYWGQGTLVTVKPGG |
| 5 | INBRX-109 | EVQLLESGGGEVQPGGSLRLSCAASGLTFPNYGMGWFRQAPGKEREFVSAIYWS |
| | VHH-linker- | GGTVYYAESVKGRFTISRDNAKNTLYLQMSSLRAEDTAVYYCAVTIRGAATQTW |
| | VHH | KYDYWGQGTLVTVKPGGSGGSEVQLLESGGGEVQPGGSLRLSCAASGLTFPNYG |
| | | MGWFRQAPGKEREFVSAIYWSGGTVYYAESVKGRFTISRDNAKNTLYLQMSSLR |
| | | AEDTAVYYCAVTIRGAATQTWKYDYWGQGTLVTVKPGG |
| 6 | Fc (with | DKTHTCPPCPAPGGPSVFLFPPKPKDTLMISRTPEVTCVVVDVSHEDPEVKENW |
| | hinge) | YVDGVEVHNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQK |
| | | SLSLSPGK |
| 7 | INBRX-109 | EVQLLESGGGEVQPGGSLRLSCAASGLTFPNYGMGWFRQAPGKEREFVSAIYWS |
| | | GGTVYYAESVKGRFTISRDNAKNTLYLQMSSLRAEDTAVYYCAVTIRGAATQTW |
| | | KYDYWGQGTLVTVKPGGSGGSEVQLLESGGGEVQPGGSLRLSCAASGLTFPNYG |
| | | MGWFRQAPGKEREFVSAIYWSGGTVYYAESVKGRFTISRDNAKNTLYLQMSSLR |
| | | AEDTAVYYCAVTIRGAATQTWKYDYWGQGTLVTVKPGGGGDKTHTCPPCPAPGG |
| | | PSVFLFPPKPKDTLMISRTPEVTCVVVDVSHEDPEVKENWYVDGVEVHNAKTKP |
| | | REEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLD |
| | | SDGSFFLYSKLTVDKSRWQQGNVFSCSVMHEALHNHYTQKSLSLSPGK |
| 8 | Human DR5 | MEQRGQNAPA ASGARKRHGP GPREARGARP GPRVPKTLVL VVAAVLLLVS |
| | (with signal | AESALITQQD LAPQQRAAPQ QKRSSPSEGL CPPGHHISED GRDCISCKYG |
| | peptide, | QDYSTHWNDL LFCLRCTRCD SGEVELSPCT TTRNTVCQCE EGTFREEDSP |
| | amino acids | EMCRKCRTGC PRGMVKVGDC TPWSDIECVH KESGTKHSGE VPAVEETVTS |
| | 1-55) | SPGTPASPCS LSGIIIGVTV AAVVLIVAVF VCKSLLWKKV LPYLKGICSG |
| | | GGGDPERVDR SSQRPGAEDN VLNEIVSILQ PTQVPEQEME VQEPAEPTGV |
| | | NMLSPGESEH LLEPAEAERS QRRRLLVPAN EGDPTETLRQ CEDDFADLVP |
| | | FDSWEPLMRK LGLMDNEIKV AKAEAAGHRD TLYTMLIKWV NKTGRDASVH |
| | | TLLDALETLG ERLAKQKIED HLLSSGKFMY LEGNADSAMS |
| 9 | Mature | ITQQD LAPQQRAAPQ QKRSSPSEGL CPPGHHISED GRDCISCKYG |
| | human DR5 | QDYSTHWNDL LFCLRCTRCD SGEVELSPCT TTRNTVCQCE EGTFREEDSP |
| | | EMCRKCRTGC PRGMVKVGDC TPWSDIECVH KESGTKHSGE VPAVEETVTS |
| | | SPGTPASPCS LSGIIIGVTV AAVVLIVAVF VCKSLLWKKV LPYLKGICSG |
| | | GGGDPERVDR SSQRPGAEDN VLNEIVSILQ PTQVPEQEME VQEPAEPTGV |
| | | NMLSPGESEH LLEPAEAERS QRRRLLVPAN EGDPTETLRQ CFDDFADLVP |
| | | FDSWEPLMRK LGLMDNEIKV AKAEAAGHRD TLYTMLIKWV NKTGRDASVH |
| | | TLLDALETLG ERLAKQKIED HLLSSGKFMY LEGNADSAMS |

What is claimed is:

1. A method of treating cancer in a subject in need thereof, comprising administering to the subject (a) a Death Receptor 5 (DR5) agonist, wherein the DR5 agonist is tetravalent, and (b) an Inhibitor of Apoptosis Protein (IAP) antagonist.

2. The method of claim 1, wherein the DR5 agonist is a DR5-binding polypeptide.

3. The method of claim 1 or claim 2, wherein the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.

4. The method of claim 3, wherein the at least one VHH domain comprises an amino acid sequence at least 90%, at least 95%, at least 99%, or 100% identical to the amino acid sequence of SEQ ID NO: 4.

5. The method of any one of claims 2-4, wherein the DR5-binding polypeptide comprises a VHH domain comprising the amino acid sequence of SEQ ID NO: 4.

6. The method of any one of claims 2-5, wherein the DR5-binding polypeptide comprises an Fc region.

7. The method of claim 6, wherein the Fc region comprises the amino acid sequence of SEQ ID NO: 6.

8. The method of any one of claims 2-7, wherein the DR5-binding polypeptide has the structure VHH-linker-VHH-linker-Fc.

9. The method of any one of claims 2-8, wherein each VHH domain comprises a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.

10. The method of any one of claims 2-9, wherein the VHH-linker-VHH comprises an amino acid sequence at least 90%, at least 95%, at least 99%, or 100% identical to the amino acid sequence of SEQ ID NO: 5.

11. The method of claim 10, wherein the VHH-linker-VHH comprises the amino acid sequence of SEQ ID NO: 5.

12. The method of any one of claims 2-11, wherein the DR5-binding polypeptide comprises an amino acid sequence at least 90%, at least 95%, at least 99%, or 100% identical to the amino acid sequence of SEQ ID NO: 7.

13. The method of any one of claims 2-12, wherein the DR5-binding polypeptide comprises the amino acid sequence of SEQ ID NO: 7.

14. The method of any one of claims 2-12, wherein the DR5-binding polypeptide consists of the amino acid sequence of SEQ ID NO: 7.

15. The method of claim 1 or claim 2, wherein the DR5 agonist is INBRX-109.

16. The method of any one of claims 1-15, wherein the IAP antagonist is a small molecule.

17. The method of any one of claims 1-16, wherein the IAP antagonist is APG-1387 (Ascentage Pharma Group International), birinapant (IGM Biosciences, Inc.), AZD5582 (AstraZeneca), LCL161 (Novartis), Debio 1143 (Merck, Debiopharm), or ASTX660 (Astex Pharmaceuticals, Inc.).

18. The method of claim 17, wherein the IAP antagonist is APG-1387.

19. The method of claim 17, wherein the IAP antagonist is birinapant.

20. The method of claim 17, wherein the IAP antagonist is Debio 1143.

21. The method of claim 17, wherein the IAP antagonist is LCL161.

22. The method of any one of claims 1-21, wherein the DR5 agonist and the IAP antagonist are administered separately.

23. The method of claim 22, wherein the DR5 agonist and the IAP antagonist are administered sequentially.

24. The method of claim 22 or 23, wherein at least one dose, or the first dose, of the DR5 agonist is administered before the IAP antagonist.

25. The method of claim 22 or 23, wherein at least one dose, or the first dose, of the DR5 agonist is administered after the IAP antagonist.

26. The method of any one of claims 1-21, wherein the DR5 agonist and the IAP antagonist are administered concurrently.

27. The method of any one of claims 1-26, wherein the DR5 agonist and the IAP antagonist act synergistically.

28. The method of claim 27, wherein synergy is determined in an in vitro cell survival assay.

29. The method of any one of claims 1-28, wherein administration of the DR5 agonist and the IAP antagonist results in a synergistic effect, compared to each agent administered alone.

30. The method of any one of claims 1-29, wherein the cancer is adrenal cancer; astrocytoma; basal cell carcinoma, biliary tract cancer; bladder cancer; bone cancer; brain and central nervous system cancer; breast cancer; cancer of the peritoneum; cervical cancer; choriocarcinoma; chondrosarcoma; Ewing sarcoma; colon and rectum cancer (colorectal cancer); connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; leukemia; liver cancer; lung cancer; small-cell lung cancer; non-small cell lung cancer; adenocarcinoma of the lung; squamous carcinoma of the lung; melanoma; myeloma; neuroblastoma; oral cavity cancer (lip, tongue, mouth, and/or pharynx); ovarian cancer; pancreatic cancer, such as pancreatic adenocarcinoma; pituitary gland cancer; prostate cancer; retinoblastoma; rhabdomyosarcoma; rectal cancer; cancer of the respiratory system; mesothelioma; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

31. A DR5 agonist for use in a method of treating cancer, wherein the method comprises administering the DR5 agonist in combination with an IAP antagonist.

32. Use of a DR5 agonist for the manufacture of a medicament for treating cancer, wherein the medicament is for administration with an IAP antagonist.
